(19) United States
(12) Patent Application Publication
(10) Pub. No.: US 2019/0113523 A1
(43) Pub. Date: Apr. 18, 2019

(54) HUMORAL IMMUNITY SIGNATURES FOR ANTIBODY-MEDIATED IMMUNE RESPONSES AND TREATMENTS

(71) Applicant: THE GENERAL HOSPITAL CORPORATION, Boston, MA (US)

(72) Inventor: Galit ALTER, Winchester, MA (US)

(73) Assignee: THE GENERAL HOSPITAL CORPORATION, Boston, MA (US)

(21) Appl. No.: 16/093,839

(22) PCT Filed: Apr. 19, 2017
(86) PCT No.: PCT/US2017/028368

§ 371 Date: Oct. 15, 2018

Related U.S. Application Data
(60) Provisional application No. 62/324,543, filed on Apr. 19, 2016.

(51) Int. Cl. G01N 33/68; C07K 16/00

(52) U.S. Cl. CPC G01N 33/6854; C07K 16/00; G01N 2800/26; G01N 2333/162; G01N 2800/52

(57) ABSTRACT

Embodiments herein provide methods for identifying antibody glycosylation profiles which correlate with antibody effector functions. These profiles are antibody signatures for the respective correlated antibody effector functions, and are incorporated into engineered antibody for antibody-mediated treatments of infections, diseases, and medical conditions.

### CROSS REFERENCE TO RELATED APPLICATION

This application claims benefit under 35 U.S.C. § 119(e) of the U.S. provisional application No. 62/324,543 filed Apr. 19, 2016, the contents of which are incorporated herein by reference in its entirety.

### TECHNICAL FIELD

The disclosed technology relates to antibody glycosylation, antibody effector functions, and antibody-mediated treatments of infections and diseases.

### BACKGROUND

There is an enormous heterogeneity in the effector functions of antibody responses to an antigen in individuals. For example, during vaccination with an immunogen, e.g., hepatitis B virus (HBV), some individuals would go on to produce antibodies and become protected against the HBV while others do not. Even within those individuals who generate antibodies, the antibodies exhibit a gradation of effector functions in clearing the HBV, thereby protecting the vaccinated individual. Understanding the differences in the effector functions of antibody responses to an antigen and also the underlying reasons for the differences is needed and can help develop novel antibody-mediated treatments for infections and diseases.

### SUMMARY

Embodiments of the present disclosure are based, in part, on the discovery that certain antibodies found in infected or diseased subjects correlated with some positive clinical outcome of the infection or disease in the subjects. Specifically, these antibodies have unique effector functional activities against the target antigen, the activities are driven or governed by particular glycosylation profiles on the antibodies. For example, as described in the Example section, in HIV-infected subjects, the subjects with immunoglobulins G (IgGs) having the following glycosylation profiles, G2, G2S, or G2S1F, have a faster clearance of serum viremia while being treated with an anti-viral retroviral therapy and also have a longer viral rebound time when the anti-viral retroviral therapy was withdrawn. The particular glycosylation profiles are useful as indicators or signatures of the respective positive clinical outcomes of infections and diseases, for prognosing the efficacy of treatments of infections and diseases, and for guiding the development of synthetic monoclonal antibodies having the particular glycosylation signature for use in the treatments of the respective infections and diseases.

The disclosure provides methods for screening and discovering unique glycosylation profiles of antibodies generated in vivo during an infection or disease wherein the glycosylation profiles correlated with a positive clinical outcome of the infection or disease in the afflicted subject. The antibodies are directed at target antigens that are known to be associated with the infection or disease. For example, a target antigen for hepatitis B infection is the hepatitis B surface antigen (HBsAg), which is a protein on the surface of HBV. HBsAg is the antigen used to make hepatitis B vaccine. For example, a target antigen associated with the human immunodeficiency virus (HIV) infection is the HIV viral protein surface glycoprotein-120 (gp120) which is one of the glycoprotein found on the HIV virus envelop. An HIV-positive subject would have anti-gp120 antibodies in them. Another target antigen for an HIV infection is HIV viral protein surface glycoprotein-41 (gp41). An HIV-positive subject would have anti-gp41 antibodies in them.

Additionally, the disclosure also provide methods for screening and discovering unique glycosylation profiles of antibodies generated in vivo to unknown target antigens. For example, a library of possible antigens that is used to elicit an immune response, e.g., a heat-killed pathogen or an attenuated pathogen. For example, in the generation of monoclonal antibodies for research, analytical, and diagnostic purposes. The generated antibodies can be analyzed for their glycosylation profiles and their different effector functions in vitro. Various antibody effector functions are known in the art and are also discussed herein. A positive correlation of a particular antibody glycosylation profile with an effective effector function in vitro is the aim of the screen. That particular antibody glycosylation profile would be selected for use in designing into monoclonal antibodies that target the antigen. That is, the identified antibody glycosylation profile is a signature that is used to guide the generation of monoclonal antibody with better effector functions, both in vitro and in vivo.

The present disclosure also provides methods for synthesizing engineered monoclonal antibodies having the desired glycosylation signatures of positive clinical outcomes of infections and diseases. The monoclonal antibodies are designed to have a particular glycosylation signature or profile that has been shown to correlate with a particular positive clinical outcome of a respective infection or disease. It is contemplated that the use of such engineered monoclonal antibodies to treat the respective infection or disease would aid bringing forth the particular positive clinical outcome of the infection or disease.

The present disclosure also provides treatment methods for an infection or a disease in an afflicted subject by administering a therapeutically effective amount of the engineered monoclonal antibodies having desired glycosylation signatures of positive clinical outcomes of infections and diseases.

Provided herein is a method of screening for and identifying a humoral immunity signature for a clinical outcome of an infection, a disease or a condition in a subject, the method comprising: (a) providing an antibody sample obtained from the subject; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the disease or condition. Typically, a population of subjects would be involved in such a screening study. A body of data would be collected from the population and the data would entered into a computational linear regression analysis program, e.g., the Least absolute shrinkage (LASSO) program, to sort out the data and correlations in order to identify a correlation with the state of the clinical outcome under study.

Provided herein is a method of screening for a humoral immunity signature for a clinical outcome of an infection, a disease, or a condition in a subject, the method comprising: (a) providing a plurality of antibody samples obtained from the subject over a period of time; (b) assaying a property or a characteristic of each of the plurality of antibody samples obtained from the subject in step (a); (c) determining a state of a clinical outcome in the subject at the point in time when the antibody sample was collected in step (a); (d) correlating the property or characteristic of each of the plurality of antibody samples obtained in step (b) with the state of a clinical outcome in the subject obtained in step (c); and (e) determining the presence of a positive or negative correlation which is indicative of a humoral immunity signature for the clinical outcome of the disease or condition. Generally, the collection of data over a period of time allows one to study the progression of the infection, disease or condition in the afflicted subject, whether the subject is treated or now. A state of a clinical outcome can be the rate of progression of the infection, disease or condition, with or without treatment. Another state of a clinical outcome can be the rate of recovery from the infection, disease or condition, with or without treatment.

Provided herein is a method of synthesizing engineered monoclonal antibodies for use with an antibody-mediated immune response/treatment to an infection, a disease, or a condition, the engineered monoclonal antibodies having a humoral immunity signature in the form of desired glycosylation in the Fc region of the antibodies, the signature correlates with a positive clinical outcome of infections, diseases, or condition, the method comprising: (a) providing an antibody sample obtained from the subject; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); (e) determining the presence of a positive or negative correlation which is indicative of a humoral immunity signature for the clinical outcome of the disease or condition; and (f) synthesizing engineered monoclonal antibodies having the identified signature. The desired antibody glycosylation profile is a signature that is used to guide the generation of monoclonal antibody with better effector functions. The desired antibody glycosylation state that is designed into the engineered monoclonal antibodies directs the antibody effector functions in vivo. In some embodiments, the antibody effector functions are the anti-pathogen or disease killing functions of antibodies known in the art.

Provided herein is a method of treating an infection, a disease, or a condition in a subject comprising administering a therapeutically effective amount of engineered monoclonal antibodies having a humoral immunity signature in the form of desired glycosylation in the Fc region of the antibodies, where the signature correlates with a positive clinical outcome of the infection, disease, or condition. The treated subject would experience a positive clinical outcome of the infection, disease, or condition.

Provided herein is a method of treating an infection, a disease, or a condition in a subject comprising administering a therapeutically effective amount of a composition comprising engineered monoclonal antibodies having a humoral immunity signature in the form of desired glycosylation in the Fc region of the antibodies, where the signature correlates with a positive clinical outcome of the infection, disease, or condition. The treated subject would experience a positive clinical outcome of the infection, disease, or condition.

In one embodiment of any one of the method described, the method further comprising using the antibody signature to design a monoclonal therapeutic for use in the treatment of the disease or condition. For example, using the information of the signature, and design and synthesizing antibodies that would have that same signature. It in contemplated that the antibody glycosylation profile is a signature that direct the desired anti-pathogen or disease killing functions (antibody effector functions) of the antibody, thus the incorporation of the antibody glycosylation profile into the monoclonal antibody generated would produce engineered antibodies with better effector functions.

In one embodiment of any one of the method described, the humoral immunity signature is found on the Fc region of the antibody, e.g., the constant region of the heavy and/or light chains of the antibody.

In one embodiment of any one of the method described, the humoral immunity signature is a functional property, or a biophysical characteristic, or both a functional property and biophysical characteristic of an antibody in the subject.

In one embodiment of any one of the method described, non-limiting examples of a state of clinical outcome of an infection, a disease, or a condition are parasite load, pathogen load, virus load, bacteria load, fungal load, disease or condition symptom(s), tumor size, rate of tumor shrinkage, rate of tumor growth, rate of cancer metastasis, neuromuscular electrical conduction, rate of loss of neuromuscular electrical conduction, cognitive performance, rate of deterioration of cognitive performance, rebound viremia, size of viral reservoir, and length of protection (vaccine), the rate of progression of the infection, disease or condition, with or without treatment, the rate of recovery from the infection, disease or condition, with or without treatment, and altered glycosylation in both O-linked and N-linked glycans in cancer progression.

In one embodiment of any one of the method described, non-limiting examples of the clinical outcome of an infection, a disease, or a condition are faster rate parasite clearance, faster tumor remission, faster tumor regression, faster pathogen (e.g., parasites, virus, bacteria) clearance, faster elimination of clinical symptoms of the disease or condition, faster rate of pathogen eradication, faster rate of elimination of CCC-DNA, and slower rate of disease progression or symptoms. For example, presence of altered glycosylation in both O-linked and N-linked glycans in cancer progression.

In one embodiment of any one of the method described, the subject has been treated for the infection, disease, or condition or is being treated for the infection, disease, or condition.

In one embodiment of any one of the method described, the subject is not being treated for the infection, disease, or condition.

In one embodiment of any one of the method described, non-limiting examples of the infection, disease, or condition are malaria, merkell cell carcinoma, nasopharyngeal carcinoma, post-transplant lymphoproliferative disease (PTLD), burkitt's lymphoma, kaposi's sarcoma, drug resistant CMV-CMV colitis, CMV hepatitis, genital/oral lesions, hepatitis, cervical cancer, invasive fungal infection, non-tubercuolosis mycobacterial infection, extended spectrum beta-lactamase producer, Alzheimer's disease, multiple schlerosis, typhoid fever, and HIV infection.

In one embodiment of any one of the method described, the invasive fungal infection is caused by Aspegillus sp. or Mucor sp.

In one embodiment of any one of the method described, the non-tubercuolosis mycobacterial (NTM) infection is caused by Mycobacterium sp. bacteria.

In one embodiment of any one of the method described, the Mycobacterium sp. bacteria is selected from the group consisting of Mycobacterium avium, Mycobacterium intracellulare, Mycobacteriu abscessus, and Mycobacterium kansasii.

In one embodiment of any one of the method described, the extended spectrum beta-lactamase producer is caused by gram-negative bacteria selected from the group selected from Escherichia coli, Klbsiella sp., Pseudomonas sp., and Neisseria gonorrhea.

In one embodiment of any one of the method described, the biophysical assays performed for the antibody samples comprises antibody isotyping subclass analysis, Fc-receptor binding assay, and glycosylation analysis of the Fc region of the antibody.

In one embodiment of any one of the method described, the glycosylation analysis of the Fc region comprises analysis for galactosylation, sialation, bisecting GlcNAc-N-acetyleglucosamine, manosylation, N-acetylegalactosamine, glucosylation and/or fucosylation.

In one embodiment of any one of the method described, the functional property assay performed for the antibody samples are antibody effector function assays.

In one embodiment of any one of the method described, the non-limiting examples of functional property assay performed for the antibody samples are antibody dependent NK cell activation (ADNKA); antibody-dependent cellular cytotoxicity (ADCC); antibody-dependent cellular phagocytosis (ADCP); antibody-dependent complement deposition (ADCD); antibody-dependent neutrophil activation/phagocytosis (ADNP); antibody dependent macrophage phagocytosis (ADMP); antibody dependent dendritic cell (DC) phagocytosis (ADDCP); antibody-dependent mucin binding (ADMB); antibody-dependent eosinophil degranulation (ADED); and antibody-dependent basophil degranulation (ADBD).

In one embodiment of any one of the method described, the antibody is an IgG, IgA, IgE, IgD, or IgM antibody.

In one embodiment of any one of the method described, the antibody is obtained from the plasma or serum of the subject.

In one embodiment of any one of the method described, the antibody is directed to a target antigen. Contemplated target antigens are any entity that can elicit an immune response in a subject where antibodies are generate and the antibodies are directed the entity. The entity is specific to an infection, disease or condition. For examples, target antigens are present in tumors, pathogens, and autoimmune conditions. For examples, target antigens are glycans, altered glycans and/or lipids.

In one embodiment of any one of the method described, the target antigen is specific to the respective infection, disease, or condition.

In one embodiment of any one of the method described, non-limiting examples of a target antigen are proteins, glycoproteins, peptides, sugars, carbohydrates, glycans, lipids and nucleic acids specific to the respective infection, disease, or condition. For example, a surface glycoprotein from the HIV or the lipopolysaccharides from the Typhoid bacteria.

In one embodiment of any one of the method described, the property assay is performed with an antigen specific to the respective infection, disease or condition.

In one embodiment of any one of the method described, the antibody is antigen-specific to the respective infection, disease, or condition.

In one embodiment of any one of the method described, the method further comprising entering the data collected into a computational linear regression analysis program to perform the correlation analysis, e.g., into the Least absolute shrinkage (LASSO) program, to sort out the data and correlations, in order to identify a correlation with the state of the clinical outcome under study.

In one embodiment of any one of the method described, the positive or negative correlation has at least an absolute R value of greater than 0.6.

### BRIEF DESCRIPTION OF THE DRAWINGS

FIGS. 1A-1K collectively show the extra-neutralizing effector functions induced by HIV gp120 specific IgG are associated with viral rebound and decrease of the HIV reservoir.

FIG. 1A shows the study overview. Shaded area indicates panobinostat treatment period.

FIG. 1B shows the exemplary Whisker box plots and line graphs depicting HIV gp120 antibody-directed effector functional profiles over time. The box-plots (upper row) depict the raw data starting at baseline (B) and at follow-up time-points (v6, v9 and v12) (ANOVA with Bonferroni adjusted p-values). The line plots (row below) shows fold-changes from baseline (Wilcoxon signed-rank test).

FIGS. 1C-1F show the Spearman's rank correlation analyses indicating the relationship between Neutrophil phagocytosis (ADNP) or complement C3b-deposition (ADCD) and changes in total HIV DNA from (FIG. 1C and FIG. 1D) baseline to either late PNB treatment (_{Δ}V9), or FIGS. 1E and 1F) throughout the entire study period (β). The change in HIV DNA dependent on increase in ADNP and ADCD, stratified by the median. The correlation plot represents the spearman's rank correlation between changes in the effector functions and decline in total HIV DNA using the linear regression coefficient (β).

FIGS. 1G and 1H show the changes in HIV DNA during panobinostat dosing between the “high” and “low” groups are shown in the dot plot, determined by the median value of ADNP β or ADCD β (Mann-Whitney).

FIG. 1I is a plot showing the increase in effector function profile from baseline to panobinostat dosing (_{Δ}V9) using the linear regression coefficient (β) describing changes in HIV DNA, during the study period, split in “high” and “low” groups by the median (low group indicating the biggest decline in HIV DNA).

FIG. 1J shows the Spearman's rank correlation between HIV gp120 specific IgG induced ADNP and time to viral rebound.

FIG. 1K shows the heat map depicting pairwise correlations between gp120 IgG titers and effector functions. Increasing shading in the box with asterisks indicates increasing positive correlation, while increasing shading without asterisks indicates negative. * p<0.05; **p<0.01.

FIGS. 2A-2E collectively show that glycosylation of HIV-1 gp120 specific IgG predicts time to viral rebound and decrease in the HIV-1 reservoir size. HIV-1 gp120 specific IgG was isolated and glycans profiled by capillary electrophoresis.

FIG. 2A shows the line plots represent relative changes (Δ) in non-galactosylated (G0), monogalactosylated (G1) and digalatosylated (G2) HIV-1 gp120 specific IgG from baseline to v6, v9 and v12.

FIG. 2B shows correlation plots depicting the relationships between the glycosylated antibody profile changes over time and viral rebound after treatment interruption or HIV DNAβ.

FIG. 2C show the hierarchical clustering of IgG-G2 temporal dynamics and viral rebound time demonstrate a clear unsupervised split in antibody profiles among subjects that rebound rapidly and slowly.

FIG. 2D shows the Kaplan-Meier curve that represents the time to rebound among subjects with high or low IgG-G2β glycoform levels (HR: 2.73 [95% CI: 2.6-43.2]).

FIG. 2E shows dot plots of IgG-G2β high or low groups show the differences in geometric mean of DNA relative to baseline (left panel) and HIV DNAβ (right panel).

FIGS. 3A-3C collectively show that multidimensional analysis identifies both IgG major glycosylation groups and specific derived functions associated with HIV DNA changes, and time to rebound.

FIG. 3A shows the minimal features required to separate the multivariate profiles were determined by LASSO, identifying 8 out of 198 features (the right panel) that best separated the groups of HIV DNA responder (red) and non-responder (blue) as well as the corresponding viral rebound rates according to PCA analysis (the left panel). Together, LV1 and LV2 captured 72.1% of the variance in the X-axis and 9% of the variance is captured in the Y-axis.

FIG. 3B shows the VIP-scores from the LASSO-PCA.

FIG. 3C shows the temporal correlations of G2 and sialic acid with effector functions. ADNP was the only function that positively correlated with G2 and sialic acid, while the other functions were inversely correlated.

FIG. 3D shows that the correlation network depicts the multidimensional relationships between antibody responses, HIV DNA changes, and time to rebound. Nodes represent all analyzed features. The linkages represent significant correlations (adjusted p value<0.05) and the color represent the direction of the correlation (blue=anti-correlated and red=correlated).

FIGS. 4A-4F collectively demonstrate the specific glycan substructures on HIV-1 gp120 specific IgG that is associated with both function and rebound time.

FIG. 4A shows that during panobinostat treatment, stepwise multiple regression identified just 3 substructures (G2SF1, G2SF and G2S1FB) together predicting rebound time by 98%.

FIG. 4B shows a Kaplan-Meier curve indicating the time to rebound among subjects with high or low IgG-G2S1F substructure levels during panobinostat treatment.

FIG. 4C shows that the same substructure (G2S1F) was correlated to change in HIV DNA at the same time point (Spearman).

FIG. 4D shows that the same substructure (G2S1F) was correlated to change in ADNP at the same time point (Spearman).

FIGS. 4E-4F show the role of individual glycoforms (non-galactosylated (G0), galactosylation with (S) or without (NS) sialic acid) was assessed on a single monoclonal PGT121 background in driving ADNP (FIG. 4E) or ADNP (FIG. 4F) in the presence of human complement. (mean [±SEM]).

FIG. 4G shows the VIP scores predicting rebound during PNB treatment.

FIG. 4H shows the glycosylation patterns of the different PGT121 glycoconstructs.

FIGS. 5A-5C collectively demonstrate that glycan substructures predicts rebound time across independent HIV cohorts.

FIGS. 5A and 5B show the independent cohort study results of patients who have received ART before treatment interruption. Glycan substructures, individually weakly associated with rebound time, were identified in both a different HIV cohort undergoing treatment interruption, but not treated with any HDAC inhibitor.

FIG. 5C is a ROC curve showing cross-prediction analysis finding the substructures in one cohort to predict rebound time in the other cohort, and vice versa, with 75% accuracy.

FIG. 6 shows a highly galactosylated, sialylated and fucosylated HIV-specific monoclonal antibody drives more efficient elimination of HIV-infected primary CD4+ T cells compared to the same monoclonal HIV-specific antibody with wild-type glycosylation. An HIV specific monoclonal antibody targeting gp120 was generated in a 293T cell line (wildtype) or a 293T cell that overexpresses B4GALT1 (galactose-transferase), ST6GAL1 (sialic acid transferase), and FUT8 (fucose transferase) resulting in the generation of highly galactosylated, sialatylated and fucosylated antibodies were incubated with HIV-infected primary CD4+ T cells from 3 different donors in the presence of neutrophils. The level of HIV-infected CD4+ T cells were then analyzed by targeted HIV Gag quantitative PCR. The addition of the glycan modified antibody resulted in a significant reduction in HIV-infected CD4+ T cells compared to the same unmodified antibody.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Michael R. Green and Joseph Sambrook, Molecular Cloning: A Laboratory Manual, Cold Spring Harbor Laboratory Press, Cold Spring Harbor, N.Y., USA (2012); Sambrook et al., Molecular Cloning: A Laboratory Manual (4th ed.); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Meolcular Biology, Vo. 203, 2003, Transgenic Mouse, editored by Marten H. Hofker and Jan van Deursen, which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments of the present disclosure are based, in part, on the identification of unexpected humoral biomarkers that can predict the time line to viral rebound in HIV-infected subjects. Specifically these antibodies have particular glycosylation profiles. In a specific antigen-specific (gp120) antibody, the glycosylation profile enhances neutrophil and complement activity, and the profile has a distinct glycan modification on antigen-specific antibodies: G2S1F. In the Example section, the anti-retroviral therapy was withdrawn from the HIV-infected subjects and the reactivation of the HIV viral reservoir was initiated with panobinostat. Antibody samples were isolated from these subjects over time and analysed. In addition, HIV DNA and serum viremia in the subjects were also analyzed. The subjects with IgGs having the following glycosylation profiles, G2, G2S, or G2S1F, have a faster clearance of serum viremia while being treated with an anti-viral retroviral therapy, and also have a longer viral rebound time when the anti-viral retroviral therapy was withdrawn. Humoral glycosylation profiles are useful as indicators or signatures of the respective positive clinical outcomes of infections and diseases, for prognosing the efficacy of treatments of the infections and diseases, and for the guiding the development of synthetic monoclonal antibodies having the particular glycosylation signature for use in the treatments of the respective infections and diseases.

Accordingly, in one embodiment, provided herein is a method of screening for a humoral immunity signature for a clinical outcome of a disease or condition in a subject, the method comprising: (a) providing an antibody sample obtained from the subject; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the disease or condition. Ideally, antibody samples are collected from a population of subjects who are afflicted with the disease or condition, e.g., hepatitis A, and the clinical outcome is a complete cure from the hepatitis A viral infection.

In another embodiment, provided herein is a method of screening for a humoral immunity signature for a clinical outcome of a disease or condition in a subject, the method comprising: (a) providing a plurality of antibody samples obtained from the subject over a period of time; (b) assaying a property or a characteristic of each of the plurality of antibody samples obtained from the subject in step (a); (c) determining a state of a clinical outcome in the subject at the point in time when the antibody sample was collected in step (a); (d) correlating the property or characteristic of each of the plurality of antibody samples obtained in step (b) with the state of a clinical outcome in the subject obtained in step (c); and (e) determining the presence of a positive or negative correlation which is indicative of a humoral immunity signature for the clinical outcome of the disease or condition. In one embodiment, the period of time can be a few weeks, such as, 5 weeks, 8 weeks, 12 weeks, 16 weeks, 20 weeks, 5 months, 8 months, 10 months or 12 months.

In one embodiment, provided herein is a method of screening for a humoral immunity signature for a clinical outcome of malaria in a subject, the method comprising: (a) providing an antibody sample obtained from individual subjects from a population of subjects who have been infected with malaria; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of malaria. In one embodiment, the population of subjects have been successfully treated for the intracellular parasitic infection. In one embodiment, the state of the clinical outcome is a complete cure or eradication of the parasite Plasmodium spp. and the total time to complete eradication of the parasite from the start of treatment. In one embodiment, the property or characteristic of the antibody is plotted against the total time to complete eradication of the parasite from the start of treatment, and the correlation is calculated therefrom. In one embodiment, the antibody sample from the subject comprises an antibody against an antigen associated with malaria, such as the histidine-rich protein 2 (HRP-2) or glutamate-rich polypeptide (GLURP) from Plasmodium falciparum, and parasite-specific lactate dehydrogenase (pLDH). For additional malaria antigens, see U.S. Pat. Nos. 4,707,357; 4,886,782; 5,231,168; and 6,942,866, the contents of each are incorporated by reference herein in their entirety.

In one embodiment, provided herein is a method of screening for a humoral immunity signature for a clinical outcome of a viral infection in a subject, the method comprising: (a) providing an antibody sample obtained from individual subjects from a population of subjects who have been infected with the viral infection; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the viral infection. In one embodiment, non-limiting examples of the viral infection are disclosed in Table 2, e.g., merkell virus, Ebstein Bar virus, HHV8, cytomegalovirus, HSV1 and HSV2, hepatitis B virus, human immunodeficiency virus (HW), a human T-lymphotrophic virus (HTLV), a herpes virus, an Epstein-Barr virus, or a human papilloma virus.

Immunosuppression of a host immune response plays a role in persistent infection and tumor immunosuppression. Persistent infections are infections in which the virus is not cleared but remains in specific cells of infected individuals. Persistent infections often involve stages of both silent and productive infection without rapidly killing or even producing excessive damage of the host cells. There are three types of persistent virus-host interaction: latent, chronic and slow infection. Latent infection is characterized by the lack of demonstrable infectious virus between episodes of recurrent disease. Chronic infection is characterized by the continued presence of infectious virus following the primary infection and can include chronic or recurrent disease. Slow infection is characterized by a prolonged incubation period followed by progressive disease. Unlike latent and chronic infections, slow infection may not begin with an acute period of viral multiplication. During persistent infections, the viral genome can be either stably integrated into the cellular DNA or maintained episomally. Persistent infection occurs with viruses such as human T-Cell leukemia viruses, Epstein-Barr virus, cytomegalovirus herpesviruses, varicella-zoster virus, measles, papovaviruses, xenotropic murine leukemia virus-related virus (XMRV), prions, hepatitis viruses, adenoviruses, parvoviruses and papillomaviruses.

The mechanisms by which persistent infections are maintained can involve modulation of virus and cellular gene expression and modification of the host immune response. Reactivation of a latent infection may be triggered by various stimuli, including changes in cell physiology, superinfection by another virus, and physical stress or trauma. Host immunosuppression is often associated with reactivation of a number of persistent virus infections.

Many studies show defective immune responses in patients diagnosed with cancer. A number of tumor antigens have been identified that are associated with specific cancers. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the innate immune system of these patients often fails to respond to the tumor antigens.

Immunosuppression of a host immune response plays a role in persistent infection and tumor immunosuppression. Persistent infections are infections in which the virus is not cleared but remains in specific cells of infected individuals. Persistent infections often involve stages of both silent and productive infection without rapidly killing or even producing excessive damage of the host cells. There are three types of persistent virus-host interaction: latent, chronic and slow infection. Latent infection is characterized by the lack of demonstrable infectious virus between episodes of recurrent disease. Chronic infection is characterized by the continued presence of infectious virus following the primary infection and can include chronic or recurrent disease. Slow infection is characterized by a prolonged incubation period followed by progressive disease. Unlike latent and chronic infections, slow infection may not begin with an acute period of viral multiplication. During persistent infections, the viral genome can be either stably integrated into the cellular DNA or maintained episomally. Persistent infection occurs with viruses such as human T-Cell leukemia viruses, Epstein-Barr virus, cytomegalovirus, herpesviruses, varicella-zoster virus, measles, papovaviruses, xenotropic murine leukemia virus-related virus (XMRV), prions, hepatitis viruses, adenoviruses, parvoviruses and papillomaviruses.

The mechanisms by which persistent infections are maintained can involve modulation of virus and cellular gene expression and modification of the host immune response. Reactivation of a latent infection may be triggered by various stimuli, including changes in cell physiology, superinfection by another virus, and physical stress or trauma. Host immunosuppression is often associated with reactivation of a number of persistent virus infections.

Many studies show defective immune responses in patients diagnosed with cancer. A number of tumor antigens have been identified that are associated with specific cancers. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the innate immune system of these patients often fails to respond to the tumor antigens.

In one embodiment, the population of subjects have been successfully treated for the viral infection. In one embodiment, the population of subjects continuing being treated for the viral infection. In one embodiment, the population of subjects have been successfully treated for the cancer that arises from the viral infection. See Table 2. In one embodiment, the state of the clinical outcome is a complete cure or eradication of the causative viral agent. In another embodiment, the state of the clinical outcome is the total time to complete cure or eradication of the causative viral agent from the start of treatment. In another embodiment, the state of the clinical outcome is a reduction in the viral load. In another embodiment, the state of the clinical outcome is a reduction of the tumor or remission of the cancer. In one embodiment, the property or characteristic of the antibody is plotted against any of the disclosed states of clinical outcomes disclosed here, and the correlation is calculated therefrom. In one embodiment, the antibody sample from the subject comprises an antibody against an antigen associated with the viral infection or the cancer arising from the viral infection, such as the Large T tumor antigen of the Merkel Cell Polyomavirus, or a tumor-associated antigen such as PRAME, WT1, Survivin, cyclin D, cyclin E, proteinase 3 and its peptide PR1, neutrophil elastase, cathepsin G, MAGE, MART, tyrosinase, GP100, NY-Eso-1, herceptin, carcino-embryonic antigen (CEA), or prostate specific antigen (PSA). For additional viral infection or tumor-associated antigens, see U.S. Pat. Appl. No: US20110135598; US20110182901; and US20100151492, the contents of each are incorporated by reference herein in their entirety.

In one embodiment, provided herein is a method of screening for a humoral immunity signature for a clinical outcome of a fungal infection in a subject, the method comprising: (a) providing an antibody sample obtained from individual subjects from a population of subjects who have been infected with the fungal infection; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the fungal infection. In one embodiment, non-limiting examples of the fungal infection are disclosed in Table 2, and Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and Trichophyton faviforme. Other contemplated fungal pathogens comprise Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp.

In one embodiment, the population of subjects have been successfully treated for the fungal infection. In one embodiment, the population of subjects continuing being treated for the fungal infection. In one embodiment, the state of the clinical outcome is a complete cure or eradication of the causative fungal agent. In another embodiment, the state of the clinical outcome is the total time to complete cure or eradication of the causative fungal agent from the start of treatment. In another embodiment, the state of the clinical outcome is a reduction in the fungal infection. In one embodiment, the property or characteristic of the antibody is plotted against any of the disclosed states of clinical outcomes disclosed here, and the correlation is calculated therefrom. In one embodiment, the antibody sample from the subject comprises an antibody against an antigen associated with the causative fungal agent, e.g., the mannan antigen of Candida albicans and the fungal galactomannan-like antigens. For additional fungal infection or tumor-associated antigens, see U.S. Pat. Appl. No: US20040152649; and US20040022869, the contents of each are incorporated by reference herein in their entirety.

In one embodiment, provided herein is a method of screening for a humoral immunity signature for a clinical outcome of a bacterial infection in a subject, the method comprising: (a) providing an antibody sample obtained from individual subjects from a population of subjects who have been infected with the bacterial infection; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the bacterial infection. In one embodiment, non-limiting examples of the bacterial infection are disclosed in Table 2, gram positive bacteria such as Pasteurella species, Staphylococci species, and Streptococcus species, gram negative bacteria such as Escherichia coli, Psuedomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In one embodiment, the population of subjects have been successfully treated for the bacterial infection. In one embodiment, the population of subjects continuing being treated for the bacterial infection. In one embodiment, the state of the clinical outcome is a complete cure or eradication of the causative bacterial agent. In another embodiment, the state of the clinical outcome is the total time to complete cure or eradication of the causative fungal agent from the start of treatment. In another embodiment, the state of the clinical outcome is a reduction in the bacterial infection. In one embodiment, the property or characteristic of the antibody is plotted against any of the disclosed states of clinical outcomes disclosed here, and the correlation is calculated therefrom. In one embodiment, the antibody sample from the subject comprises an antibody against an antigen associated with the causative bacterial agent include but are not limited to the major secreted (MPT32, MPT44, MPT46, MPT51, MPT53, MPT59, MPT63, and MPT64) and somatic mycobacterial antigens (Mce1A, Hsp65, and MPT57), an iron-regulated outer membrane protein, (IROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia. For additional bacterial antigens, see U.S. Pat. Appl. No: US20040152649; and US20040022869, the contents of each are incorporated by reference herein in their entirety.

In one embodiment, provided herein is a method of screening for a humoral immunity signature for a clinical outcome of a HIV infection in a subject, the method comprising: (a) providing an antibody sample obtained from individual subjects from a population of HIV-positive subjects; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the HIV infection. In one embodiment, the population of subjects have been treated for the HIV infection and there is no detectable virus in blood circulation. In one embodiment, the population of subjects are continuing being treated for the HIV infection even when there is non-detectable viremia, that is the presence of a virus in the blood. In one embodiment, the state of the clinical outcome is a complete, non-detectable viremia. In another embodiment, the state of the clinical outcome is the total time to complete non-detectable viremia from the start of treatment. In another embodiment, the state of the clinical outcome is the time to detectable viremia after the withdrawal of anti-retroviral treatment. In another embodiment, the state of the clinical outcome is the time to detectable viremia after the withdrawal of anti-retroviral treatment and the reactivation of latent viral reservoir. In one embodiment, the property or characteristic of the antibody is plotted against any of the disclosed states of clinical outcomes disclosed here, and the correlation is calculated therefrom. In one embodiment, the antibody sample from the subject comprises an antibody against an antigen associated with the HIV include but are not limited to the p24 and the glycoprotein gp120. Antibodies against the HIV-1 and HIV-2 are known in the art.

In one embodiment, provided herein is a method of screening for a humoral immunity signature for a clinical outcome of an autoimmune disease in a subject, the method comprising: (a) providing an antibody sample obtained from individual subjects from a population of subjects having the autoimmune disease; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the autoimmune disease. In one embodiment, non-limiting examples of the autoimmune diseases are disclosed in Table 2.

The term “autoimmune disease” refers to those disease states and conditions wherein the immune response of the patient is directed against the patient's own constituents, resulting in an undesirable and often terribly debilitating condition. As used herein, “autoimmune disease” is intended to further include autoimmune conditions, syndromes and the like. An “autoantigen” is a patient's self-produced constituent, which is perceived to be foreign or undesirable, thus triggering an autoimmune response in the patient, which may in turn lead to a chain of events, including the synthesis of other autoantigens or autoantibodies. An “autoantibody” is an antibody produced by an autoimmune patient to one or more of his own constituents which are perceived to be antigenic. For example, in AIDS disease the patient eventually produces autoantibodies to CD4 cells, in SLE autoantibodies are produced to DNA, while in many other types of AD autoantibodies are produced to target cells.

Patients suffering from autoimmune diseases including, e.g., rheumatoid arthritis, insulindependent diabetes mellitus, hemolytic anemias, rheumatic fever, thyroiditis, Crohn's disease, myasthenia gravis, glomerulonephritis, autoimmune hepatitis, multiple sclerosis, systemic lupus erythematosus and others.

In one embodiment, the population of subjects have been successfully treated for the autoimmune disease. In one embodiment, the population of subjects are continuing being treated for the autoimmune disease. In one embodiment, the state of the clinical outcome is a complete remission of the autoimmune disease without treatment. In another embodiment, the state of the clinical outcome is the total time to complete remission of the autoimmune disease from the start of treatment. In another embodiment, the state of the clinical outcome is a normalized score for a symptom of the autoimmune disease. In another embodiment, the state of the clinical outcome is the period of time remained in remission before a relapse of autoimmune disease symptoms. In one embodiment, the property or characteristic of the antibody is plotted against any of the disclosed states of clinical outcomes disclosed here, and the correlation is calculated therefrom. In one embodiment, the antibody sample from the subject comprises an antibody against an autoantigen, e.g., tau, amyloid, and myelin.

Having identified the specific humoral immunity signature of the antibody for a desired clinical outcome of an infection, disease or condition, e.g., malaria, HIV infection, other viral infections, cancers, bacterial infection, fungal infections, and autoimmune diseases, the information of the identified signature can be used to design and synthesize monoclonal antibodies for use in the treatment of the respective infection, disease or condition.

Accordingly, in one embodiment, provided herein is a method of synthesizing engineered monoclonal antibodies for use with an antibody-mediated immune response treatment to an infections, diseases, or condition, the engineered monoclonal antibodies having a humoral immunity signature in the form of desired glycosylation in the Fc region of the antibodies, the signature correlates with a positive clinical outcome of infections, diseases, or condition, the method comprising: (a) providing an antibody sample obtained from the subject; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); (e) determining the presence of a positive or negative correlation which is indicative of a humoral immunity signature for the clinical outcome of the disease or condition; and (0 synthesizing engineered monoclonal antibodies having the identified signature. In some embodiments, the infections, diseases, or conditions are pathogen infection, microbial infection, malaria, HIV infection, other viral infections, cancers, bacterial infection, fungal infections, and autoimmune diseases. In some embodiments, the state of a clinical outcome include but is not limited to a complete, non-detectable viremia, the total time to complete non-detectable viremia from the start of treatment, the time to detectable viremia after the withdrawal of anti-retroviral treatment, the time to detectable viremia after the withdrawal of anti-retroviral treatment and the reactivation of latent viral reservoir, a complete remission of the autoimmune disease without treatment, the total time to complete remission of the autoimmune disease from the start of treatment, a normalized score for a symptom of the autoimmune disease, the period of time remained in remission before a relapse of autoimmune disease symptoms, a complete cure or eradication of the causative bacterial agent, the total time to complete cure or eradication of the causative fungal agent from the start of treatment, a reduction in the bacterial infection, a complete cure or eradication of the parasite Plasmodium spp. and the total time to complete eradication of the parasite from the start of treatment, a complete cure or eradication of the causative viral agent, the total time to complete cure or eradication of the causative viral agent from the start of treatment, a reduction in the viral load, a reduction of the tumor or remission of the cancer, a complete cure or eradication of the causative fungal agent, the total time to complete cure or eradication of the causative fungal agent from the start of treatment, and a reduction in the fungal infection.

The described engineered antibody can be synthesized by any method known in the art. For examples, as described in the U.S. Pat. Nos. 5,545,403; 6,254,868; 6,358,710; 6,602,684; U.S. Pat. Appl. No: US20030175884; US20110207676; and in the International Pat. Appl. Publication No: WO 2017/044850, the contents of each are incorporated by reference herein in their entirety.

In another embodiment, provided herein is a method of treating an infections, diseases, or condition in a subject comprising administering a therapeutically effective amount of engineered monoclonal antibodies having a humoral immunity signature in the form of a desired antibody glycosylation state in the Fc region of the antibodies, where the signature correlates with a positive clinical outcome of infections, diseases, or condition. In some embodiment, the route of administration can be any route that is suitable for treating the infections, diseases, or condition in a subject.

In another embodiment, provided herein is a use of an engineered monoclonal antibody having a humoral immunity signature in the form of a desired antibody glycosylation state in the Fc region of the antibody for the treatment of an infection, disease, or condition, where the signature correlates with a positive clinical outcome of infection, disease, or condition.

In another embodiment, provided herein is an engineered monoclonal antibody having a humoral immunity signature in the form of a desired antibody glycosylation state in the Fc region of the antibody for use in the treatment of an infection, disease, or condition, where the signature correlates with a positive clinical outcome of infection, disease, or condition.

In another embodiment, provided herein is an engineered monoclonal antibody having a humoral immunity signature in the form of a desired antibody glycosylation state in the Fc region of the antibody for use in the manufacture of a medicament for the treatment of an infection, disease, or condition, where the signature correlates with a positive clinical outcome of infection, disease, or condition.

The synthesized engineered antibodies are useful in a variety of applications including, but not limited to, therapeutic treatment methods, such as the treatment of viral infections, bacterial infections, parasitic infections and cancer/tumors. Examples of viral infections include, but are not limited to, Human Immunodeficiency Virus (HIV), Herpes Simplex Virus (HSV), Epstein-Barr Virus (EBV), Cytomegalovirus (CMV), Herpes Simplex, Influenza (flu), Ebola Virus Disease (EVD)/Ebola hemorrhagic fever (EHF), and Hepatitis C. Examples of bacterial infections include, but are not limited to, Tuberculosis (TB), Strep Throat, Meningitis and Pneumonia. Examples of parasitic infections include, but are not limited to, Malaria, Amoebiasis, Giardiasis, Leishmaniasis, and Toxoplasmosis. Examples of cancer include, but are not limited to, ovarian cancer, pancreatic cancer and prostate cancer.

Terms such as “treating” or “treatment” or “to treat” or “alleviating” or “to alleviate” refer to therapeutic treatment and/or prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. Thus, those in need of treatment include those already with the disorder; those prone to have the disorder; and those in whom the disorder is to be prevented.

The term “subject” refers to any animal (e.g., a mammal), including, but not limited to humans, non-human primates, rodents, and domestic and game animals, which is to be the recipient of a particular treatment. Primates include chimpanzees, cynomologous monkeys, spider monkeys, and macaques, e.g., Rhesus. Rodents include mice, rats, woodchucks, ferrets, rabbits and hamsters. Typically, the terms “subject” and “patient” are used interchangeably herein in reference to a human subject. In various embodiments, a subject can be one who has been previously diagnosed with or identified as suffering from or having a condition in need of treatment. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition.

The term “therapeutically effective amount” refers to an amount of an antibody, polypeptide, polynucleotide, small organic molecule, or other drug effective to “treat” a disease or disorder in a subject or mammal. The therapeutically effective amount of the drug can reduce the severity of the disease symptoms, including HIV symptoms. These include, but are not limited to, diarrhea, fever, fatigue, abdominal pain, abdominal cramping, inflammation, nausea, vomiting, reduced appetite, and weight loss.

In another embodiment, provided herein is a composition comprising an engineered monoclonal antibody having a humoral immunity signature in the form of a desired antibody glycosylation state in the Fc region of the antibody, where the signature correlates with a positive clinical outcome of infections, diseases, or condition. In one embodiment, the composition further comprises a pharmaceutically acceptable carrier. In some embodiments, the composition is formulated for administration in a route that is suitable for treating the infections, diseases, or condition in a subject.

In one embodiment of any one of the method described, the method further comprising using the signature to design a monoclonal therapeutic for use in the treatment of the disease or condition. For example, using the information of the signature, and design and synthesizing antibodies that would have that same signature.

In one embodiment of any one of the method described, the humoral immunity signature is found on the Fc region of the antibody.

In one embodiment of any one of the method described, the humoral immunity signature is the antibody glycosylation state on the Fc region of the antibody.

In one embodiment of any one of the method described, the humoral immunity signature is a functional property, a biophysical characteristic, or a combination of both a functional property and biophysical characteristic of an antibody in the subject.

In one embodiment of any one of the method described, the state of clinical outcome of a disease or condition is selected from the group consisting of parasite load, pathogen load, virus load, bacteria load, fungal load, disease or condition symptom(s), tumor size, neuromuscular electrical conduction, cognitive performance, rebound viremia, size of viral reservoir, and length of protection (vaccine).

In one embodiment of any one of the method described, non-limiting examples of the clinical outcome of a disease or condition include faster rate parasite clearance, faster tumor remission, faster tumor regression, faster pathogen (e.g., parasites, virus, bacteria) clearance, faster elimination of clinical symptoms of the disease or condition, faster rate of pathogen eradication, faster rate of elimination of CCC-DNA, and slower rate of disease progression or symptoms.

In one embodiment of any one of the method described, the subject has been treated for the disease or condition or is being treated for the disease or condition. In one embodiment, the treatment has been successful and the subject is no longer being treated. In one embodiment, the treatment has been successful in getting rid of active pathogen but the subject continues with a latent pathogen load, e.g., in a hidden reservoir, e.g., in HIV infection.

In one embodiment of any one of the method described, the disease or condition is selected from the group consisting of malaria, merkell cell carcinoma, nasopharyngeal carcinoma, post-transplant lymphoproliferative disease (PTLD), burkitt's lymphoma, kaposi's sarcoma, drug resistant CMV-CMV colitis, CMV hepatitis, genital/oral lesions, hepatitis, cervical cancer, invasive fungal infection, non-tubercuolosis mycobacterial infection, extended spectrum beta-lactamase producer, Alzheimer's disease, multiple schlerosis, typhoid fever, and HIV infection.

In one embodiment of any one of the method described, the disease or condition is a bacterial infection, a viral infection, a fungal infection, and a parasite infection.

In one embodiment of any one of the method described, the disease or condition is active tuberculosis (TB).

In one embodiment of any one of the method described, the disease or condition is associated with elderly flu, syphilis, legionella, lyme disease and/or CMV reactivation.

In one embodiment of any one of the method described, the viral infection is selected from the group consisting of Human immunodeficiency viruses (HW-1 and HW-2), Human T lymphotrophic virus type I (HTLV-I), Human T lymphotrophic virus type II (HTLV-II), Herpes simplex virus type I (HSV-1), Herpes simplex virus type 2 (HSV-2), Human papilloma virus (multiple types), Hepatitis A virus, Hepatitis B virus, Hepatitis C and D viruses, Epstein-Barr virus (EBV), Cytomegalovirus and Molluscum contagiosum virus. In one embodiment, the viral infection is a herpes simplex virus infection. In one embodiment, the viral infection is HIV-1 or HIV-2.

In one embodiment of any one of the method described, the invasive fungal infection is caused by Aspegillus sp. or Mucor sp.

In one embodiment of any one of the method described, the non-tubercuolosis mycobacterial (NTM) infection is caused by Mycobacterium sp. bacteria.

In one embodiment of any one of the method described, the Mycobacterium sp. bacteria is selected from the group consisting of Mycobacterium avium, Mycobacterium intracellulare, Mycobacteriu abscessus, and Mycobacterium kansasii.

In one embodiment of any one of the method described, the extended spectrum beta-lactamase producer is caused by gram-negative bacteria selected from the group selected from Escherichia coli, Klbsiella sp., Pseudomonas sp., and Neisseria gonorrhea.

In other embodiments, the infection is an infection with a microbial species selected from the group consisting of herpesviridae, retroviridae, orthomyroviridae, toxoplasma, haemophilus, campylobacter, clostridium, E. coli, and staphylococcus.

In one embodiment of any one of the method described, the biophysical assays performed for the antibody samples comprises antibody isotyping subclass analysis, Fc-receptor binding assay, and glycosylation analysis of the Fc region of the antibody.

In one embodiment of any one of the method described, the glycosylation analysis of the Fc region comprises analysis for galactosylation, sialation, bisecting GlcNAc-n-acetyleglucosamine, manosylation, n-acetylegalactosamine, glucosylation and/or fucosylation.

In one embodiment of any one of the method described, the functional property assay performed for the plurality of antibody samples is selected from the group consisting of Antibody dependent NK cell activation (ADNKA); Antibody-dependent cellular cytotoxicity (ADCC); Antibody-dependent cellular phagocytosis (ADCP); Antibody-dependent complement deposition (ADCD); Antibody-dependent neutrophil activation/phagocytosis (ADNP); Antibody dependent macrophage phagocytosis (ADMP); Antibody dependent dendritic cell (DC) phagocytosis (ADDCP); Antibody-dependent mucin binding (ADMB); Antibody-dependent eosinophil degranulation (ADED); and Antibody-dependent basophil degranulation (ADBD).

In one embodiment of any one of the method described, the antibody is an IgG, IgA, IgE, IgD, or IgM antibody.

In one embodiment of any one of the method described, the antibody is obtained from the plasma or serum of the subject.

In one embodiment of any one of the method described, the property assay is performed with an antigen specific to the disease or condition.

In one embodiment of any one of the method described, the antibody is antigen specific to the disease or condition.

In one embodiment of any one of the method described, the method further comprising entering the data collected into a computational regression analysis program to perform a correlation analysis, e.g., into the Least absolute shrinkage (LASSO) program, to sort out the data and correlations, in order to identify a correlation with the state of the clinical outcome under study.

In one embodiment of any one of the method described, the data for input into the computational regression analysis program are the results of functional assays and the biophysical assays of the antibody samples, the state of the clinical outcomes, and the clinic outcomes. These data for the variables in the multivariable regression analysis.

In one embodiment of any one of the method described, the data collected is integrated in a systems biology analysis aimed at identifying the minimal functional and biophysical signatures that predict the positive clinical outcomes, e.g., disease control, which is then used to design an antigen specific antibody. Where novel antigen-specific antibodies may be generated to the desired target OR existing antibodies to that target are used to engineer the Fc-domain of the antibody to accelerate the development of therapeutics with the target product profile predicted to exhibit enhanced clinical efficacy.

In one embodiment of any one of the method described, the data collected is integrated into a multi-variable regression analysis. Regression analysis methods are known in the art. For example, see WO 2017/044850, the contents are incorporated by reference is its entirety. Non-limiting multi-variable regression analysis methods include the Least absolute shrinkage (LASSO) program which is an L1-constrained fitting program for statistics and data mining; R Companion program for multiple logistic regression; and multiple logistic regression in SAS.

In statistics and machine learning, lasso (least absolute shrinkage and selection operator) (also Lasso or LASSO) is a regression analysis method that performs both variable selection and regularization in order to enhance the prediction accuracy and interpretability of the statistical model it produces. It minimizes the usual sum of squared errors, with a bound on the sum of the absolute values of the coefficients. It has connections to soft-thresholding of wavelet coefficients, forward stagewise regression, and boosting methods. It is used in a wide variety of statistical models including generalized linear models, generalized estimating equations, proportional hazards models, and M-estimators, in a straightforward fashion. See the internet webpage “The Lasso Page.”

In one embodiment of any one of the method described, the computational regression analysis program analyze the multivariable data obtained and is used to define the minimal information related to the “correlate” or “signature.” In other words, the minimal information produced by the computational regression analysis program in the antibody glycosylation profile that correlates with the clinical outcome focused in the screening method.

In one embodiment of any one of the method described, the positive or negative correlation has at least an absolute R value of greater than 0.6.

In one embodiment, as used herein, the term “immunogen” or “antigen” or “target antigen” as used interchangeably. In one embodiment, as used herein, the term “immunogen” or “antigen” or “target antigen” as used hereinafter comprises any entity capable of producing a protective antibody or cell-mediated immunological response against a pathogenic organism in an animal. The antigen or immunogen can be whole pathogen or part of the pathogen including any cell component, a protein, glycoprotein, glycolipid, polysaccharide, lipopolysaccharide or nucleic acid which belongs or is associated with the pathogen, or it may be a polypeptide or other entity which mimics all or part of such a pathogen or protein, glycoprotein, glycolipid, polysaccharide, lipopolysaccharide or nucleic acid thereof. An “antigen” as used herein is a molecule capable of provoking an immune response. Antigens include but are not limited to cells, cell extracts, proteins, polypeptides, peptides, lipids, glycans, polysaccharides, polysaccharide conjugates, peptide and non-peptide mimics of polysaccharides and other molecules, small molecules, glycolipids, carbohydrates, nucleic acids, viruses and viral extracts and multicellular organisms such as parasites and allergens. The term antigen broadly includes any type of molecule which is recognized by a host immune system as being foreign. In one embodiment, the antigen is associate with a disease or condition. For example, multiple sclerosis and cancer. Antigens include but are not limited to cancer antigens, microbial antigens, and allergens.

The terms glycan and polysaccharide are defined by IUPAC as synonyms meaning “compounds consisting of a large number of monosaccharides linked glycosidically”. However, in practice the term glycan may also be used to refer to the carbohydrate portion of a glycoconjugate, such as a glycoprotein, glycolipid, or a proteoglycan, even if the carbohydrate is only an oligosaccharide. Glycans usually consist solely of 0-glycosidic linkages of monosaccharides. For example, cellulose is a glycan (or, to be more specific, a glucan) composed of β-1,4-linked D-glucose, and chitin is a glycan composed of β-1,4-linked N-acetyl-D-glucosamine. Glycans can be homo- or heteropolymers of monosaccharide residues, and can be linear or branched.

A “microbial antigen” as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

A “cancer antigen” as used herein is a compound, such as a peptide, protein, glycans, lipids, and lipids present in a tumor or cancer cell and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor, cancer cells, or cancer cell lysate. Such antigens can be isolated or prepared recombinantly or by any other means known in the art.

Cancer or tumor antigens are differentially expressed by cancer cells and can thereby be exploited in order to target cancer cells. Some of these antigens are encoded, although not necessarily expressed, by normal cells. Most cancer antigens are altered glycans, for example, altered glycosylation in both O-linked and N-linked glycans is known in cancer progression. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

Target antigens or immunogens can be derived from various sources including tumor, non-tumor cancers, allergens, and infectious pathogens: bacteria, viruses, fungi, and parasites. Each of the lists recited herein is not intended to be limiting.

Examples of viruses that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

Polypeptides of bacterial pathogens include but are not limited to an iron-regulated outer membrane protein, (IROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordain; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of Ichthyophthirius. Also contemplated are lipids, glycans, and lipopolysaccharides of a parasitic pathogen

Examples of fungi include Cryptococcus neoformans, Histoplasma capsulatum, Coccidioides immitis, Blastomyces dermatitidis, Chlamydia trachomatis, Candida albicans. Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia micron, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii. Other medically relevant microorganisms have been described extensively in the literature, e.g., see C. G. A Thomas, Medical Microbiology, Bailliere Tindall, Great Britain 1983, the entire contents of which is hereby incorporated by reference.

The methods of use may be in vitro, ex vivo, or in vivo methods. In certain embodiments, the disease treated with the synthesized engineered antibodies is HIV.

In various embodiments, the synthesized engineered antibodies described herein can be formulated for delivery via any route of administration. “Route of administration” refers to any administration pathway known in the art, including but not limited to, transmucosal, transdermal or parenteral.

“Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the enteral route, the synthesized engineered antibodies described herein can be in the form of tablets, gel capsules, sugar-coated tablets, syrups, suspensions, solutions, powders, granules, emulsions, microspheres or nanospheres or lipid vesicles or polymer vesicles allowing controlled release.

In various embodiments, the engineered antibodies described herein can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. In particular embodiments, compounds used herein are administered orally, intravenously or intramuscularly to a patient.

In various embodiments, the engineered antibodies described herein is a monoclonal antibody, human antibody, humanized antibody or a neutralizing antibody. The synthesized antibodies can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, the synthesized engineered antibodies including a pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in the therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain minor amounts of auxiliary substances such as wetting or emulsifying agents, pH buffering agents and the like which enhance or maintain the effectiveness of the active ingredient. The therapeutic composition as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of an active agent used in the invention that will be effective in the treatment of a particular disorder or condition will depend on the nature of the disorder or condition, and can be determined by one of skill in the art with standard clinical techniques.

The synthesized engineered antibodies described herein can be delivered in a therapeutically effective amount. The precise therapeutically effective amount is that amount of the synthesized immunogen and/or antibodies that will yield the most effective results in terms of efficacy of treatment in a given subject. The therapeutically effective amount of synthesized immunogen will induce an immune response in the subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

For the treatment of the disease, the appropriate effective amount of the synthesized engineered antibodies described herein depends on the type of disease to be treated, the severity and course of the disease, the responsiveness of the disease, and the desired clinical outcome, previous therapy, and patient's clinical history. The dosage can also be adjusted by one of skill in the art in the event of any complication and at the discretion of a person skilled in the art. The person skilled in the art can determine optimum dosages, dosing methodologies and repetition rates. The synthesized immunogen and/or antibodies can be administered one time or over a series of treatments lasting from several days to several months, or until a cure is effected or a diminution of the disease state is achieved (e.g., treatment or amelioration of the disease). The duration of treatment depends upon the subject's clinical progress and responsiveness to therapy. In certain embodiments, dosage is from 0.01 μg to 100 mg per kg of body weight, and can be given once or more daily, weekly, monthly or yearly. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In various other embodiments, the synthesized engineered antibodies described herein are administered in a series of treatments. In selected embodiments, the synthesized engineered antibodies described herein are administered to patients who have previously undergone a treatment. In some embodiments, the treatment is administered in any order, including prior to, concurrently with, substantially simultaneously or subsequent the administration of a second treatment.

### Biophysical Assays

In one embodiment of any one of the method described, one or more of the biophysical assays described herein are performed for the antibody sample collected from the subject.

In one embodiment of any one of the method described, one or more of the biophysical assays described herein are performed for each of the plurality of antibody samples collected from the subject.

In one embodiment of any one of the method described, all of the biophysical assays described herein are performed for each of the plurality of antibody samples collected from the subject.

In one embodiment of any one of the method described, all of the biophysical assays described herein are performed for the antibody sample collected from the subject.

In one embodiment of any one of the method described, the biophysical assay is isotyping of the antibody. Antibodies are glycoproteins that bind specific antigens. They are produced in response to invasion by foreign molecules in the body. Antibodies exist as one or more copies of a Y-shaped unit, composed of four polypeptide chains. Each Y contains two identical copies of a heavy chain, and two identical copies of a light chain, which are different in their sequence and length. The top of the Y shape contains the variable region, which binds tightly and specifically to an epitope on the antigen.

The light chains of an antibody can be classified as either kappa (κ) or lambda (λ) type based on small differences in polypeptide sequence. The heavy chain makeup determines the overall class of each antibody. In mammals, antibodies are divided into five isotypes: IgG, IgM, IgA, IgD and IgE, based on the number of Y units and the type of heavy chain. The isotypes differ in their biological properties, functional locations and ability to deal with different antigens. The type of heavy chain present defines the class of an antibody. There are five types of mammalian Ig heavy chain denoted by Greek letters: α, δ, ε, γ and μ. These chains are found in IgA, IgD, IgE, IgG and IgM antibodies, respectively. Heavy chains differ in size and composition; α and γ contain approximately 450 amino acids, while μ and ε have approximately 550 amino acids.

Each heavy chain has two regions, the constant region and the variable region. The constant region is identical in all antibodies of the same isotype, but differs in antibodies of different isotypes. Heavy chains γ, α and δ have a constant region composed of three tandem Ig domains and a hinge region for added flexibility, heavy chains μ and ε have a constant region composed of four immunoglobulin domains. The variable region of the heavy chain differs depending on the B cell that produced it hut is the same for all antibodies produced by a single B cell or B cell clone. The variable region of each heavy chain is approximately 110 amino acids long and is composed of a single Ig domain.

In mammals there are only two types of light chain, λ and κ. A light chain has two successive domains: one constant domain and one variable domain. The approximate length of a light chain is 211-217 amino acids. Each antibody contains two light chains that are always identical.

F(ab) and Fc regions—The Y-shape of an antibody can be divided into three sections: two F(ab) regions and an Fc region. The F(ab) regions contain the variable domain that binds to cognate antigens. The Fc fragment provides a binding site for endogenous Fc receptors on the surface of lymphocytes, and is also the site of binding for secondary antibodies. In addition, dye and enzymes can be covalently linked to antibodies on the Fc portion of the antibody for experimental visualization.

These three regions can be cleaved into two F(ab) and one Fc fragments by the proteolytic enzyme pepsin. Antibody fragments have distinct advantages in certain immunochemical techniques. Fragmenting IgG antibodies is sometimes useful because F(ab) fragments (1) will not precipitate the antigen and (2) will not be bound by immune cells in live studies because of the lack of an Fc region. Often, because of their smaller size and lack of crosslinking (due to loss of the Fc region), F(ab) fragments are radiolabeled for use in functional studies. Fc fragments are often used as Fc receptor blocking agents in immunohistochemical staining.

The isotype of the isolated antibody can be determined by any methods known in the art. For example, as described in the U.S. Pat. No. 4,727,037, the contents of which is incorporated herein by reference in its entirety.

In one embodiment of any one of the method described, the biophysical assay is an Fc receptor array. The Fc receptor array assesses the Fc-associated biophysical characteristics of antigen-specific polyclonal antibody populations in a luminex based bead array, including quantifying antigen-specific antibodies and determining their subclass as well as their ability to interact with human, NHP, and mouse Fc receptors, innate immune receptors, lectin-like molecules and lectins (n=˜75 diff possible interactors). Importantly, this array offers flexibility to map both linear and conformational epitope-specific antibody profiles.

In one embodiment of any one of the method described, the biophysical assay is a glycosylation analysis. Antibody glycosylation analysis assesses differences in antibody glycosylation, known to impact antibody functionality, using a high-throughput system based on capillary electrophoresis. Antigen-specific antibodies are isolated, glycans are enzymatically released, labeled, and quantified.

In another embodiment of any one of the method described, the glycosylation analysis comprises analysis for decreased afucosylated branched glycoforms. In another embodiment, the glycosylation analysis comprises analysis for decreased 2-Galactose (G2), fucosylated G2 (G2F), fucosylated G2 with bisecting N-acetylglucosamine (G2FB), sialylated G2 (G2S), and/or sialylated G2 with bisecting N-acetylglucosamine (G2SB).

As used herein, the term “antibody glycosylation state” refers to the pattern, e.g. position, type, frequency, and/or number of glycan residues attached to an antibody and/or population of antibodies. Various glycosylation patterns are described herein. Structural details and methods of detecting such glycosylation states are known in the art, see e.g., Demchenko et al. “Handbook of Chemical Glycosylation” Wiley-VCH, 2008; Isenberg. “Abnormalities of IgG glycosylation and immunological disorders” Wiley 1996; and Al-Rubaei “Cell Engineering: Glycosylation” Springer, 2002; each of which is incorporated by reference herein in its entirety.

In some embodiments of any one of the method described, the antibody glycosylation state can be the antibody glycosylation state of an IgG antibody. For example, human IgG antibodies comprise a conserved N-glycosylation site within the CH2 domain of their Fc moieties, where the sugar side chain is attached to the asparagine 297 (Asn297) residue. The Asn297-linked carbohydrate chain consists of a common biantennary glycan structure of four N-acetylglucosamine (GlcNAc) and three mannose residues, with variable additions of fucose, galactose, sialic acid, and/or bisecting GlcNAc residues. Depending on the presence or absence of galactose on one or both arms of the glycan moiety, various antibody glycosylation states have been identified, e.g.: afucosylated branched glycoforms; 2-Galactose (G2), fucosylated G2 (G2F), fucosylated G2 with bisecting N-acetylglucosamine (G2FB), sialylated G2 (G2S), and/or sialylated G2 with bisecting N-acetylglucosamine (G2SB). In addition, O-linked glycans are also analysed, e.g., O-acetylglucosamine. In other embodiments, the antibody glycosylation state is the antibody glycosylation state of an IgM, IgA, IgE, or IgD antibody.

In some embodiments of any one of the method described, an antibody glycosylation state include decreased bi-galactosylated (G2): decreased fucosylated G2 (G2F); decreased fucosylated G2 and bisected (G2FB); bisecting N-acetylglucosamine G0) G0B; fucosylated G0 with bisecting N-acetylglucosamine (G0FB); fucosylated G0 (G0F); and 1-galactose (G1). For further discussion of IgG glycosylation, see, e.g. Jefferis “Glycosylation of Natural and Recombinant Antibody Molecules” Chapter 26 in “Glycobiology and Medicine” Axford (ed.) Springer 2005; which is incorporated by reference herein in its entirety.

In one embodiment of any one of the method described, the glycosylation analysis is performed by mass spectrometry-based glycoproteomics that is known in the art. For example, as described in the International Patent Application publication No: WO 2017/044850 and WO 2016/064955, the contents of each are incorporated by reference herein in their entirety.

In one embodiment of any one of the method described, all of the biophysical assays described herein are performed for each of the plurality of antibody samples collected from the subject.

### Antibody Functional Assays

In one embodiment of any one of the method described, the functional property assay performed for the antibody samples are antibody effector function assays.

Antibodies act by a number of mechanisms, most of which engage other arms of the immune system. Antibodies can simply block interactions of molecules or they can activate the classical complement pathway (known as complement dependent cytotoxicity or CDC) by interaction of C1q on the C1 complex with clustered antibodies. Critically antibodies also act as a link between the antibody-mediated and cell-mediated immune responses through engagement of Fc receptors. Antibodies have several modes of action: i) they can block ligand-receptor interactions; ii) cause cell lysis through activation of complement dependant cytotoxicity (CDC); iii) interact with Fc receptors on effector cells to engage antibody dependent cellular cytotoxicity; iv) signal for ingestion of a pathogen by a phagocyte. Many antibody effector functions are mediated by Fcγ-receptors.

Fc receptors (FcRs) are key immune regulatory receptors connecting the antibody mediated (humoral) immune response to cellular effector functions. Receptors for all classes of immunoglobulins have been identified, including FcγR (IgG), FcεRI (IgE), FcαRI (IgA), FcμR (IgM) and FcδR (IgD). There are three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range KD) while FcγRII and FcγRIII are low to intermediate affinity (micromolar range KD).

In antibody dependent cellular cytotoxicity (ADCC), FcvRs on the surface of effector cells (natural killer cells, macrophages, monocytes and eosinophils) bind to the Fc region of an IgG which itself is bound to a target cell. Upon binding a signalling pathway is triggered which results in the secretion of various substances, such as lytic enzymes, perforin, granzymes and tumour necrosis factor, which mediate in the destruction of the target cell. The level of ADCC effector function various for human IgG subtypes. Although this is dependent on the allotype and specific FcvR in simple terms ADCC effector function is high for human IgG1 and IgG3, and low for IgG2 and IgG4. For example, a FcγRs bind to IgG asymmetrically across the hinge and upper CH2 region. Knowledge of the binding site has resulted in engineering efforts to modulate IgG effector functions

In one embodiment of any one of the method described, one or more of the antibody function assays described are performed for each of the plurality of antibody samples collected from the subject.

In some embodiments of any one of the method described, the antibody function analyses are performed according to any known methods in the art. For example, as described in the International Patent Application publication No: WO 2016/064955, and in Chung A W, et al., 2014, Sci. Transl. Med. 6: 228-238, Barouch D H, et al., 2015, Science, 349(6245):320-4, Margaret E. Ackerman, et al., 2016, PLoS Pathog. 12(1):e1005315; and Lu L L, et al., 2016, Cel, 167(2):433-443.e14, the contents of each are incorporated by reference herein in their entirety.

In one embodiment of any one of the method described, the antibody function assay is antibody-dependent NK cell activation assay (ADNKA). Antibody dependent NK cell activation (ADNKA) assesses antigen-specific antibody-mediated NK cell activation against protein-coated plates. Antibodies are added to antigen coated ELISA plates or beads. NK cells from seronegative donors are applied, and levels of activation markers and intracellular cytokines are measured after 5 hours of incubation by flow cytometry. Alternatively, whole bacteria or infected cells may be tethered to the plates and incubated with autologous innate effectors at increasing concentrations of antibodies of interest. Additional subsets of effector cells, including unfractionated PBMCs, monocytes, and neutrophils can be tested.

In one embodiment of any one of the method described, the antibody function assay is antibody-dependent cellular cytotoxicity (ADCC). Antibody-dependent cellular cytotoxicity (ADCC) tests the ability of monoclonal antibodies to recruit NK cell lytic activity in a LDH-based lysis assay. Infected target cells or protein adsorbed target cells incubated with autologous NK cells, PBMCs, or neutrophils for 4 hours in the presence of increasing doses of the antibodies of interest. Medium alone is used as a negative control; Triton-X is used as a positive control. Supernatants are collected and release of LDH is quantified using colormetric assay.

In one embodiment of any one of the method described, the antibody function assay is antibody-dependent cellular phagocytosis (ADCP). Antibody-dependent cellular phagocytosis (ADCP) assesses the ability of patient antibodies to induce phagocytosis of antigen-functionalized fluorescent beads via Fc receptors. Streptavidin-conjugated beads are coated with biotinylated recombinant proteins of interest or infected cells and subsequently bound with patient antibodies. A monocytic cell line or primary monocytes are mixed with beads and phagocytosis is allowed to proceed overnight. The extent of phagocytosis is measured by flow cytometry.

In one embodiment of any one of the method described, the antibody function assay is antibody-dependent complement deposition (ADCD). Antibody-dependent complement deposition (ADCD) assesses the recruitment of complement component C3b on the surface of target cells. Target cells are pulsed with antigens of interest, whole bacteria, or infected cells are incubated with the test antibodies. Human complement serum is applied for 20 minutes, and the levels of C3b deposition is measured by flow cytometry.

In one embodiment of any one of the method described, the antibody function assay is antibody-dependent neutrophil activation/phagocytosis (ADNP). Antibody-dependent neutrophil activation/phagocytosis (ADNP) assesses the ability of patient antibodies to mediate phagocytosis of antigen-coated beads. Fluorescent, streptavidin-conjugated beads are coupled to biotinylated antigens of interest, bacteria, or infected cells, coated with antibodies of interest and fresh human serum as a source of complement, and overlaid on polymorphonuclear leukocytes (neutrophils) that are purified from fresh human blood. After a 1 hour incubation, supernatants are collected for analysis of secreted factors (cytokines+reactive oxygen species), and cells are fixed. The amount of phagocytosis is assessed by flow cytometry. Additionally, the formation of neutrophil extracellular traps (NETs) can be quantified by high-density fluorescence microscopy in a duplicate well, if requested.

In one embodiment of any one of the method described, the antibody function assay is antibody dependent macrophage phagocytosis (ADMP). Antibody dependent macrophage phagocytosis (ADMP) assesses the ability of patient antibodies to induce macrophage phagocytosis, macrophage activation/maturation, and cytokine release. Fluorescent streptavidin-conjugated beads are coupled to biotinylated antigens of interest, bacteria, or infected cells, and then incubated with decreasing concentrations of antibodies for 1 hour. Beads are then incubated with monocyte-derived macrophages, alveolar macrophages, or tissue resident macrophages generated from healthy donors or collected from tissue samples for 18 hours. The level of phagocytosis is measured by flow cytometry as a composite measure of both the number of beads taken up by an individual cell as well as the total number of cells that take up beads. Additionally, the maturation status of macrophages is measured by the upregulation of maturation/activation markers (CD40, CD80, CD83, CD86). Finally, qualitative differences in the capacity of antibodies to direct distinct macrophage functions are measured by luminex assay by measuring changes in cytokine production in culture supernatants.

In one embodiment of any one of the method described, the antibody function assay is antibody-dependent dendritic cell (DC) phagocytosis (ADDCP). Antibody dependent dendritic cell (DC) phagocytosis (ADDCP) assesses the ability of patient antibodies to induce DC phagocytosis, DC activation/maturation, and cytokine release. Fluorescent, streptavidin-conjugated beads are coupled to biotinylated antigens of interest, bacteria, or infected cells, and then incubated with decreasing concentrations of antibodies for 1 hour. Beads are then incubated with monocyte-derived DCs generated from healthy donors for 18 hours. The level of phagocytosis is measured by flow cytometry as a composite measure of both the number of beads taken up by an individual cell as well as the total number of cells that take up beads. Additionally, the maturation status of DCs is measured by the upregulation of maturation/activation markers (CD40, CD80, CD83, CD86). Finally, qualitative differences in the capacity of antibodies to direct distinct DC functions are measured by luminex assay by measuring changes in cytokine production in culture supernatants.

In one embodiment of any one of the method described, the antibody function assay is antibody-dependent mucin binding (ADMB). Antibody-dependent mucin binding (ADMB) measures the capacity of antibodies to trap bacteria or bacteria-infected cells in mucus proteins. Fluorescent, streptavidin-conjugated beads are coupled to biotinylated antigens of interest, bacteria, or infected cells and then incubated with decreasing concentrations of antibodies for 1 hour. Beads are then incubated with recombinant or purified native (from OVCAR or ECC1 cells) membrane-associated (mucin 1, 3, 4, 12, 13, 15, 16, 17, or 20) or secreted (mucin 2, 5AC, 5B, 6, 7, 9, or 19) mucin-coated plates. The degree of mucin trapping by antibodies is measured using high-throughput, quantitative immunofluorescent microscopy. Additionally, for a subset of cell-associated mucins, such as MUC1 and MUC16, opsonized viruses and/or beads will be incubated with confluent ECC1 cells, and the degree of trapping on native mucins will be analyzed by microscopy.

In one embodiment of any one of the method described, the antibody function assay is antibody-dependent eosinophil degranulation (ADED). Antibody-dependent eosinophil degranulation (ADED) measures the capacity of antibodies to activate eosinophil degranulation. Antibodies are added to antigen coated ELISA plates or beads. Purified eosinophils from seronegative donors are applied, and levels of activation markers and secreted cytokines/leukotrienes are measured after 5 hours by flow cytometry and luminex.

In one embodiment of any one of the method described, the antibody function assay is antibody-dependent basophil degranulation (ADBD). Antibody-dependent basophil degranulation (ADBD) measures the capacity of antibodies to activate eosinophil degranulation. Antibodies are added to antigen coated ELISA plates or beads. Purified basophils from seronegative donors are applied, and levels of activation markers and secreted cytokines/leukotrienes/histamine are measured after 5 hours by flow cytometry and luminex.

The present disclosure can be defined in any of the following numbered paragraphs:
[1] A method of screening for and identifying a humoral immunity signature for a clinical outcome of an infection, a disease or a condition in a subject, the method comprising: (a) providing an antibody sample obtained from the subject; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); and (e) determining the presence of a positive or negative correlation which is indicative of a humoral immunity signature for the clinical outcome of the disease or condition.
[2] A method of screening for a humoral immunity signature for a clinical outcome of an infection, a disease, or a condition in a subject, the method comprising: (a) providing a plurality of antibody samples obtained from the subject over a period of time; (b) assaying a property or a characteristic of each of the plurality of antibody samples obtained from the subject in step (a); (c) determining a state of a clinical outcome in the subject at the point in time when the antibody sample was collected in step (a); (d) correlating the property or characteristic of each of the plurality of antibody samples obtained in step (b) with the state of a clinical outcome in the subject obtained in step (c); and (e) determining the presence of a positive or negative correlation which is indicative of a humoral immunity signature for the clinical outcome of the disease or condition.
[3] The method of paragraph 1 or 2, further comprising using the signature to design a monoclonal therapeutic for use in the treatment of the disease or condition.
[4] A method of synthesizing engineered monoclonal antibodies for use with an antibody-mediated immune response/treatment to an infection, a disease, or a condition, the engineered monoclonal antibodies having a humoral immunity signature in the form of desired glycosylation in the Fc region of the antibodies, the signature correlates with a positive clinical outcome of infections, diseases, or condition, the method comprising: (a) providing an antibody sample obtained from the subject; (b) assaying a property or a characteristic of the antibody sample in step (a); (c) determining a state of a clinical outcome in the subject at the point in time when the antibody sample was collected in step (a); (d) correlating the property or characteristic of the antibody sample obtained in step (b) with the state of the clinical outcome in the subject obtained in step (c); (e) determining the presence of a positive or negative correlation which is indicative of a humoral immunity signature for the clinical outcome of the disease or condition; and (f) synthesizing engineered monoclonal antibodies having the identified signature.
[5] A method of treating an infection, a disease, or a condition in a subject comprising administering a therapeutically effective amount of engineered monoclonal antibodies having a humoral immunity signature in the form of desired glycosylation in the Fc region of the antibodies, where the signature correlates with a positive clinical outcome of the infection, disease, or condition.
[6] A method of treating an infection, a disease, or a condition in a subject comprising administering a therapeutically effective amount of a composition comprising engineered monoclonal antibodies having a humoral immunity signature in the form of desired glycosylation in the Fc region of the antibodies, where the signature correlates with a positive clinical outcome of the infection, disease, or condition.
[7] The method of any one of the preceding paragraphs, the method further comprising using the antibody signature to design a monoclonal therapeutic for use in the treatment of the disease or condition.
[8] The method of any one of the preceding paragraphs, wherein the humoral immunity signature is found on the Fc region of the antibody.
[9] The method of any one of the preceding paragraphs, wherein the humoral immunity signature is found on the heavy chain Fc region of the antibody.
[10] The method of any one of the preceding paragraphs, wherein the humoral immunity signature is found on the light chain Fc region of the antibody.
[11] The method of any one of the preceding paragraphs, wherein the humoral immunity signature is a functional property, or a biophysical characteristic, or both a functional property and biophysical characteristic of an antibody in the subject.
[12] The method of any one of the preceding paragraphs, wherein the subject has been treated for the infection, disease, or condition or is being treated for the infection, disease, or condition.
[13] The method of any one of the preceding paragraphs, wherein the subject is not being treated for the infection, disease, or condition.
[14] The method of any one of the preceding paragraphs, wherein the functional property assay performed for the antibody samples are antibody effector function assays.
[15] The method of any one of the preceding paragraphs, wherein the state of clinical outcome of a disease or condition is selected from the group consisting of parasite load, pathogen load, virus load, bacteria load, fungal load, disease or condition symptom(s), tumor size, neuromuscular electrical conduction, cognitive performance, rebound viremia, size of viral reservoir, and length of protection (vaccine).
[16] The method of any one of the preceding paragraphs, wherein the clinical outcome of a disease or condition is selected from the group consisting of faster rate parasite clearance, faster tumor remission, faster tumor regression, faster pathogen (e.g., parasites, virus, bacteria) clearance, faster elimination of clinical symptoms of the disease or condition, faster rate of pathogen eradication, faster rate of elimination of CCC-DNA, and slower rate of disease progression or symptoms.
[17] The method of any one of the preceding paragraphs, wherein the disease or condition is selected from the group consisting of malaria, merkell cell carcinoma, nasopharyngeal carcinoma, post-transplant lymphoproliferative disease (PTLD), burkitt's lymphoma, kaposi's sarcoma, drug resistant CMV-CMV colitis, CMV hepatitis, genital/oral lesions, hepatitis, cervical cancer, invasive fungal infection, non-tubercuolosis mycobacterial infection, extended spectrum beta-lactamase producer, Alzheimer's disease, multiple schlerosis, typhoid fever, and HIV infection.
[18] The method of paragraph 17, wherein the invasive fungal infection is caused by Aspegillus sp. or Mucor sp.
[19] The method of paragraph 17, wherein the non-tubercuolosis mycobacterial (NTM) infection is caused by Mycobacterium sp. bacteria.
[20] The method of paragraph 19, wherein the Mycobacterium sp. bacteria is selected from the group consisting of Mycobacterium avium, Mycobacterium intracellulare, Mycobacteriu abscessus, and mycobacterium kansasii.
[21] The method of paragraph 17, wherein the extended spectrum beta-lactamase producer is caused by gram-negative bacteria selected from the group selected from Escherichia coli, Klbsiella sp., Pseudomonas sp., and Neisseria gonorrhea.
[22] The method of any one of the preceding paragraphs, wherein the biophysical assays performed for the antibody samples comprises antibody isotyping subclass analysis, Fc-receptor binding assay, and glycosylation analysis of the Fc region of the antibody.
[23] The method of paragraph 22, wherein the glycosylation analysis of the Fc region comprises analysis for galactosylation, sialation, bisecting GlcNAc-n-acetyleglucosamine, manosylation, n-acetylegalactosamine, glucosylation fucosylation, decreased bi-galactosylated (G2): decreased fucosylated G2 (G2F); decreased fucosylated G2 and bisected (G2FB); bisecting N-acetylglucosamine G0) G0B; fucosylated G0 with bisecting N-acetylglucosamine (G0FB);
   fucosylated G0 (G0F); and/or 1-galactose (G1).
[24] The method of any one of the preceding paragraphs, wherein the functional property assay performed for the antibody samples are antibody effector function assays.
[25] The method of any one of the preceding paragraphs, wherein the functional property assay performed for the plurality of antibody samples is selected from the group consisting of Antibody dependent NK cell activation (ADNKA); Antibody-dependent cellular cytotoxicity (ADCC); Antibody-dependent cellular phagocytosis (ADCP); Antibody-dependent complement deposition (ADCD); Antibody-dependent neutrophil activation/phagocytosis (ADNP); Antibody dependent macrophage phagocytosis (ADMP); Antibody dependent dendritic cell (DC) phagocytosis (ADDCP); Antibody-dependent mucin binding (ADMB); Antibody-dependent eosinophil degranulation (ADED); and Antibody-dependent basophil degranulation (ADBD).
[26] The method of any one of the preceding claims, wherein the antibody is an IgG, IgA, or IgM antibody.
[27] The method of any one of the preceding paragraphs, wherein the antibody is obtained from the plasma or serum of the subject.
[28] The method of any one of the preceding paragraphs, wherein the antibody is directed to a target antigen.
[29] The method of any one of the preceding paragraphs, wherein the target antigen is specific to the respective infection, disease, or condition.
[30] The method of any one of the preceding paragraphs, wherein the target antigen are proteins, glycoproteins, peptides, sugars, carbohydrates, glycans, lipids and nucleic acids specific to the respective infection, disease, or condition.
[31] The method of any one of the preceding paragraphs, wherein the property assay is performed with an antigen specific to the disease or condition.
[32] The method of any one of the preceding paragraphs, wherein the antibody is antigen specific to the disease or condition.
[33] The method of any one of the preceding paragraphs, the method further comprising entering the data collected into a computational linear regression analysis program to perform the correlation analysis, e.g., into the Least absolute shrinkage (LASSO) program, to sort out the data and correlations, in order to identify a correlation with the state of the clinical outcome under study.
[34] The method of any one of the preceding claims, wherein the positive or negative correlation has at least an absolute R value of greater than 0.6.
[35] A method of synthesizing engineered monoclonal antibodies for use with an antibody-mediated immune response to an infections, diseases, or condition, the engineered monoclonal antibodies having a humoral immunity signature in the form of desired glycosylation in the Fc region of the antibodies, the signature correlates with a positive clinical outcome of infections, diseases, or condition, the method comprising: (a) providing an antibody sample obtained from the subject; assaying a property or a characteristic of the antibody sample in step (a); (b) determining a state of a clinical outcome in the subject at the point in time when the antibody sample was collected in step (a); (c) correlating the property or characteristic of the antibody sample obtained in step (b) with the state of the clinical outcome in the subject obtained in step (c); (d) determining the presence of a positive or negative correlation which is indicative of a humoral immunity signature for the clinical outcome of the disease or condition; and synthesizing engineered monoclonal antibodies having the identified signature.
[36] A method of treating an infections, diseases, or condition in a subject comprising administering an engineered monoclonal antibodies having a humoral immunity signature in the form of desired glycosylation in the Fc region of the antibodies, where the signature correlates with a positive clinical outcome of infections, diseases, or condition.
[37] A method of predicting a HIV rebound viremia in a subject, the method comprising: (a) at a first time point, assaying a first blood sample obtained from the subject to determine a first level of the HIV glycoprotein 120-specific IgG-G2 glycoform antibody in the subject; (b) at a second time point, assaying a second blood sample obtained from the subject to determine a second level of the HIV glycoprotein 120-specific IgG-G2 glycoform antibody in the subject, wherein the second time point is after the first time point; (c) comparing the first level with the second level for indication of a downward trend or an upward trend of the antibody titer with the progression of time wherein a downward trend of antibody titer indicates the likelihood of a HIV rebound infection; and (d) administering to the subject an anti-retro viral agent when there is a downward trend of the antibody titer, wherein administration to the subject of the anti-retroviral agent treats the HIV infection.
[38] The method of claim 37, wherein the subject has terminated an anti-retro viral therapy and there is undetectable HIV viral load serologically from the subject.
[39] The method of any one of the preceding claim, wherein the subject is treated with an agent to reactivate the latent HIV infection.
[40] The method of any one of the preceding paragraphs, wherein the anti-retroviral therapy is a highly active anti-retro viral therapy (HAART).
[41] The method of of any one of the preceding paragraphs, wherein the anti-retroviral agent is selected from the group consisting of a nucleoside, a nucleoside reverse transcriptase inhibitor (NRTI), a non-nucleoside reverse transcriptase inhibitor (NNRTI), a nucleoside analog reverse transcriptase inhibitor (NARTI), a protease inhibitor, an integrase inhibitor, an entry inhibitor, a maturation inhibitor, and combinations thereof.
[42] A method of treating a HIV infection in a subject, the method comprising administering to the subject an anti-retro viral agent when there is a downward trend of the antibody titer, wherein administration to the subject of the anti-retroviral agent treats the HIV infection.
[43] A method of treating a HIV infection in a subject, the method comprising analyzing the G2S1F glycosylation state of the anti-gp120 antibody in the subject, and administering to the subject an anti-retro viral agent when there is a downward trend of the antibody titer of G2S1F glycosylation state, wherein administration to the subject of the anti-retroviral agent treats the HIV infection.
[44] A method of treating a HIV infection in a subject, the method comprising analyzing the G2S1F glycosylation state of the anti-gp120 antibody in the subject, and administering to the subject an anti-retro viral agent when the antibody titer of G2S1F glycosylation state is below a median reference, wherein administration to the subject of the anti-retroviral agent treats the HIV infection.

This invention is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the invention described herein. Such equivalents are intended to be encompassed by the following claims.

### EXAMPLES

The major obstacle for HIV “cure” strategies relates to the resistance of a latent HIV reservoir, established upon infection, to immunologic eradication, leading to life-long infection (Finzi et al., 1999). However, over the past years, several novel approaches to reactivate the latent virus have been proposed in order to expose the reservoir to the immune system. This exposure may result in viral eradication, but most likely only in the setting of highly effective immune responses. Among these reservoir “shock” strategies, histone deacetylase (HDAC) inhibitors have been used in recent clinical trials to unravel the latent viral genome and drive viral transcription, rendering reactivated HIV-infected cells vulnerable to immune-mediated killing, although only marginal effects on the circulating viral reservoir size was shown (Archin et al., 2012; Elliott et al., 2014; Rasmussen et al., 2014; Søgaard et al., 2015; Spivak et al., 2014). However, the identification of biomarkers that either functionally account or act as a surrogate of effective reservoir control mechanisms could revolutionize our ability to develop more effective “cure” strategies. Thus, immune signatures associated with delayed viral rebound or viral load clearance are urgently needed.

While HDAC inhibitors reverse HIV latency in vivo (Archin et al., 2012; Rasmussen et al., 2014; Søgaard et al., 2015), immunological signatures have only shown weak associations with key reservoir control, but not viral rebound (Olesen et al., 2015). However, beyond the role of cellular immune responses in the control of HIV, accumulating evidence suggests that functional antibodies also contribute to viral load set point control (Fuchs et al., 2015) and elite control of HIV (Ackerman et al., 2016) via their ability to recruit the cytolytic or antiviral-functions of the innate immune system. However, whether the functional and/or biophysical characteristics of HIV-specific antibodies change upon HIV reactivation and track with reservoir control/clearance or control of viral rebound is unknown.

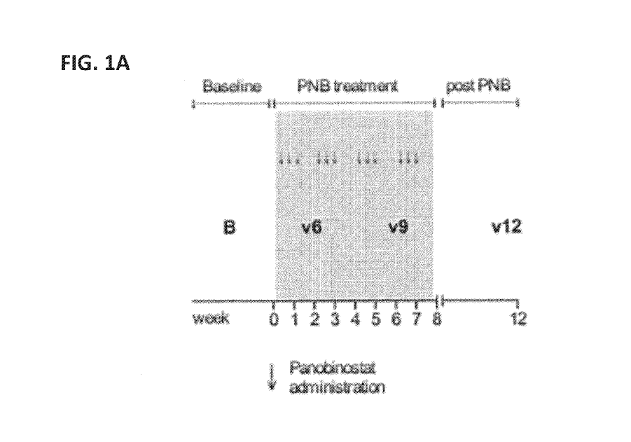

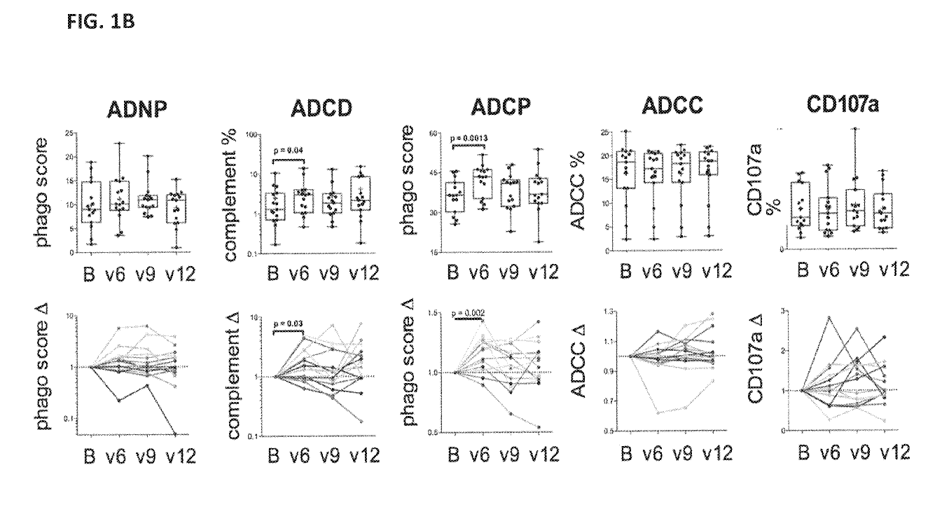

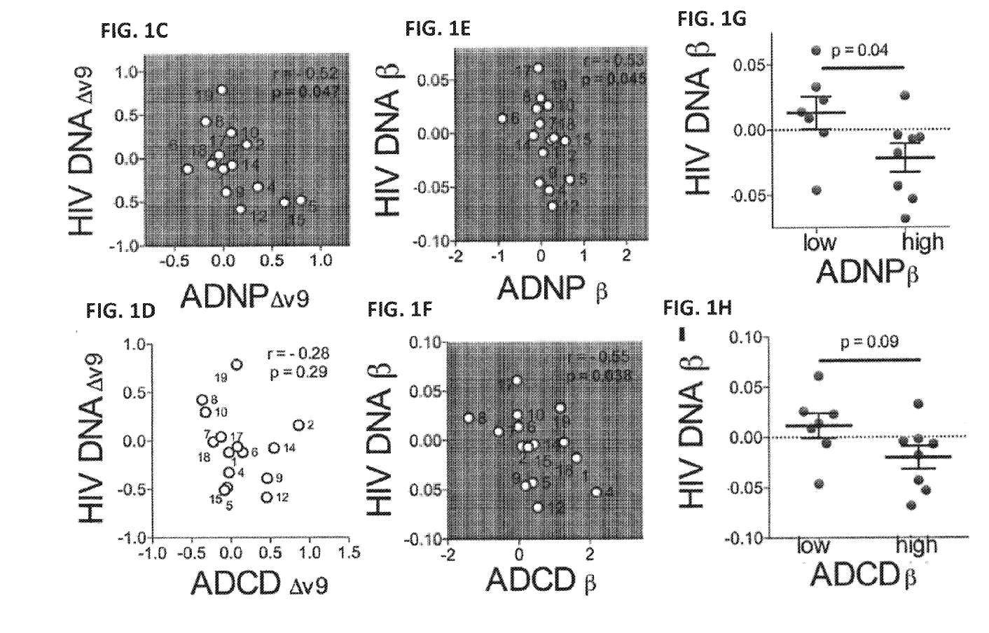

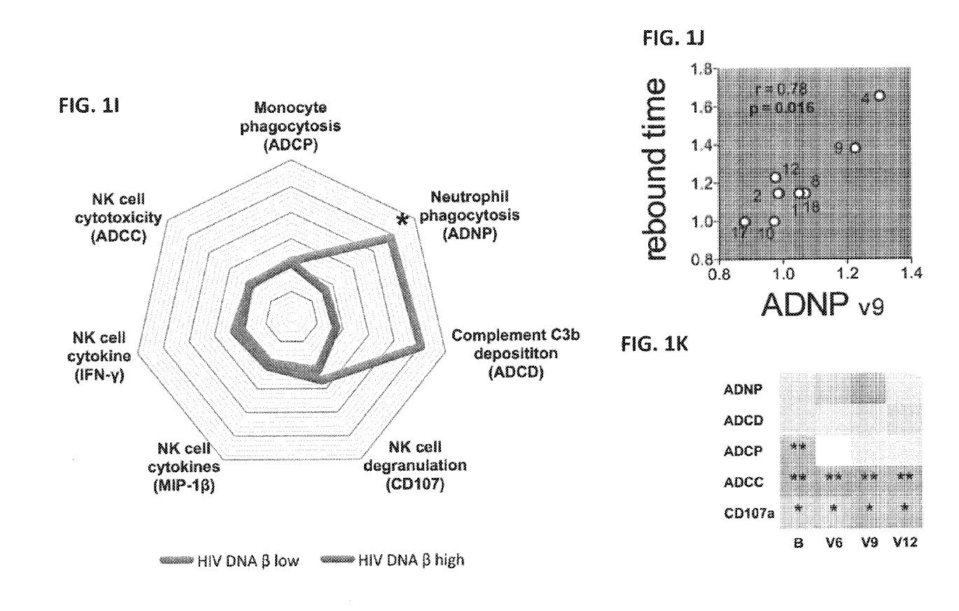

Here the functional and biophysical characteristics of HIV-specific antibodies were profiled using systems serology (Chung et al., 2015) in a clinical trial using panobinostat (PNB) as a latency-reversing agent (Rasmussen et al., 2014). Here, 15 HIV-1 infected participants received PNB over 8 weeks. Plasma samples were obtained before treatment (baseline), during PNB administraion (v6 and v9), and finally after PNB treatment (v12) (FIG. 1A). In parallel, total and integrated HIV DNA was quantified per million CD4+ T cells, along with viral outgrowth assays as different surrogate measures of the HIV viral reservoir size. At the last time point, 9 participants consented to a closely monitored analytical ART interruption, enabling the measurement of time to HIV viral rebound.

### Materials and Methods

Participants and samples—Briefly, 15 ART treated HIV-infected adults were enrolled (Rasmussen et al., 2014). Ethics committee approval was obtained in accordance with the principles of the Declaration of Helsinki. Each patient provided written informed consent. Patients received oral panobinostat (20 mg) three times per week every other week for 8 weeks. The trial was registered at ClinicalTrials.gov, identifier: NCT01680094

Antibody-Dependent Cellular Phagocytosis (ADCP)—The cellular phagocytosis assay was performed as previously described (McAndrew et al., 2011). Biotinylated recombinant HIV gp120 (sf162) (Immune Technology) was coupled to 1 μm fluorescent neutravidin beads (Invitrogen) overnight at 4° C. Beads were washed three times prior to incubation with purified IgG from individual donors in duplicate for 2 hr at 37° C. Next, monocytic THP-1 cells (ATCC) were added, and co-cultures were incubated overnight. Cells were washed and fixed in 4% PFA. Phagocytosis was measured by flow cytometry on a BD LSR II flow cytometer and a phagocytic score was calculated (frequency of bead-positive cells x MFI of bead fluorescence in same cells). Each sample was tested in two individual experiments.

Antibody-Dependent Neutrophil Phagocytosis (ADNP)—Neutrophils were isolated from whole blood from HIV-negative donor blood using dextran for 25 minutes. Supernatant was collected and remaining RBCs were lysed with dH20 for 25 seconds, followed by rapid isotonic equilibration. Biotinylated recombinant HIV gp120 (sf162) (Immune Technology) was coupled to 1 μm fluorescent neutravidin beads (Invitrogen) overnight at 4° C. Beads were washed three times before incubated with purified IgG from individual donors in duplicate for 2 hr at 37° C. Neutrophils were then added and incubated with opsonized beads for 60 minutes. Next, cells were washed and fixed in 4% PFA. Cells were stained for CD3, CD14, CD66b, CD11c (BD Biosciences). Neutrophils were defined as SSC-high, CD66b+, CD3−, CD14−, CD11c−. The degree of phagocytosis was measured by flow cytometry on a BD LSR II flow cytometer and a phagocytic score was calculated as in the ADCP assay. Each IgG sample was tested on fresh cells from two individual healthy donors, and a geometric mean score was calculated.

Antibody-dependent cellular cytotoxicity (ADCC) assay—The rapid fluorescent ADCC (RF-ADCC) assay was performed as described previously (Gómez-Róman et al., 2006). In brief, CEM-NKr cells were pulsed with recombinant HIV gp120 (sf162) and labeled with CFSE and the membrane dye PKH26. NK cells were enriched directly from seronegative whole blood donors by negative selection using RosetteSep (Stem Cell Technologies). Purified IgG was added to the labeled, antigen-pulsed CEM-NKr cells. Next, fresh NK cells were added, prior to a 4 hour incubation at 37° C. Cell were then fixed, and the proportion of cells that maintained membrane expression of PKH26 but lost CFSE staining were quantified by flow cytometry.

Antibody-dependent complement deposition (ADCD)—CEM cells were pulsed with recombinant HIV gp120 (sf162) and incubated with purified IgG from individual donors. Fifteen ul of freshly isolated, HIV negative donor plasma, diluted 1:10 with veronal buffer and 0.1% gelatin, was added before incubation for 20 minutes at 37° C. Cells were then washed with 15 mM EDTA in PBS, and complement deposition was detected via flow cytometry following staining for C3b on the surface of cells.

Antibody-dependent NK cell activation—Ab-dependent NK cell degranulation and cytokine/chemokine secretion was measured as previously described(Chung et al., 2014) with minor modifications. Briefly, CEM-NKr cells were pulsed with recombinant HIV gp120 (sf162). Fresh NK cells were isolated from whole blood from seronegative donors using negative selection with RosetteSep (StemCell). The antigen-pulsed CEM-NKr cells and isolated primary NK cells were mixed and purified Abs, anti-CD107a, brefeldin A (10 mg/ml) (Sigma), and GolgiStop (BD) were added before incubation for 5 hours at 37° C. The cells were then washed and stained for surface markers using anti-CD16, anti-CD56, and anti-CD3. The cells were then washed, fixed and permeabilized using Fix & Penn (Invitrogen), and then stained intracellularly with anti-IFN-γ and anti-MIP-1β. The cells were then fixed in 4% paraformaldehyde and analyzed on a BD LSR II flow cytometer.

Cell-associated HIV DNA—The size of the cell associated DNA reservoir was qualified as previously described (Rasmussen et al., 2014). Briefly, CD4 T cells were isolated using negative magnetic selection. Next, cells were resuspended in lysis buffer and digested. Cell lysates were used directly for quantification of HIV DNA using a QX100™ Droplet Digital™ PCR system (Bio-Rad).

Hierarchical clustering of anti-HIV-specific IgG effector functions—Hierarchical clustering was utilized to assess all effector functions from all time-points as well as changes between time-points (delta). Individual patients (depicted with ID) were arranged as endpoints in a constellation plot, with lines drawn that represent correlations between features. Longer lines represent greater distance between the clusters. For visualization of clustering of functions in the context of a viral reservoir decline, patient endpoints were labeled with colors dependent on total HIV DNAβ ‘low’ (red=HIV DNA responders) or ‘high’ (blue=HIV DNA non-responders) groups.

Luminex Isotype Assay—A luminex assay was used to quantify the relative concentration of each antibody isotype among the HIV-specific antibodies as is known in the art. Briefly, Luminex microplex carboxylated beads (Luminex) were coupled to recombinant HIV gp120 (sf162) via covalent NHS-ester linkages by combining EDC and NHS (Thermo Scientific) in PBS. The coupled beads (2500 beads/well) were added to a 96-well filter plate (Millipore). Donor plasma was added and incubated at 4° C. overnight. The beads were washed three times with 100 μl of PBS-Tween, and incubated with individual IgG isotype detection reagents (total IgG, IgG1, IgG2, IgG3, and IgG4) conjugated to PE (Southern Biotech). The 96-well plate was incubated, with shaking, for 2 hours (500 rpm), washed four times, and read on a Bio-Plex 3D Suspension Array System (Bio-Rad).

Glycan analysis of anti-HIV-specific IgG—HIV-specific antibodies were purified and Fc-antibody glycosylation was analyzed by capillary electrophoresis, as is known in the art, for examples, in Mahan et al., 2016 and Mahan et al., 2015. Briefly, plasma samples were passed over gp120 (sf162) embedded columns. The bound antibodies were eluted, treated with Ides, and the Fc portion captured by protein G beads. Glycans were released using enzymatic digestion with Peptide-N-Glycosidase F (PNGaseF, New England Biolabs). Glycan-containing supernatants were dried in a Labconco centrivap. Dried glycans were labeled with 8-aminopyrene-1,3,6-trisulfonic acid. Fluorescently labeled glycans were resuspended in ultrapure water and passed through filter plates, releasing labeled glycans in the flow-through. Glycans were stored at 4° C. until analysis on a 3130XL ABI DNA sequencer. Area under the curve for each peak was calculated using a custom designed script.

Generation of galactose PGT121-glycovariants—G0, sialylated and non-sialylated antibodies were generated by lectin-based fractionation or enzymatic cleavage of a 293T-derived PGT121 mAb preparation that expressed all antibody glycoforms. Briefly, G0-antibodies were generated from overnight incubation of PGT121 with β(1-4)-Galactosidase (BioLabs, cat.no. P0730S) and removal of free galactose sugars. In contrast, sialylated and non-sialylated G2 mAbs, were generated via the initial purification of sialated-antibodies using SNA-beads (Vector Laboratories, cat.no. AL-1303). Secondly, half of the eluate was treated with neuraminidase (BioLabs, cat.no. P0720L) to generate non-sialylated antibodies. The glycan profiles of the collected mAbs was characterized by glycan sequencing and by western hint using ECL-biotin and SNA-biotin that bind to galactose and sialic acid sugars respectively.

Construction of the correlation network—Networks were constructed based on the pairwise correlations between all the features, including antibody glycan and functional profiles, total HIV DNA, and viral rebound at each sampling time point across the 15 participants. The lines represent significant correlations between any given two nodes (i and j) with an adjusted p-value correcting for multiple hypothesis testing (False discovery rate adjusted p-value <0.05). In addition, the edge weights between nodes were determined by the soft thresholding Aij, driving from the correlation coefficient pij as follows, Aij=pij α with α=5, capturing both agonistic (positive-) and antagonistic (negative-correlations).

Identification of the viral rebound-specific correlates with LASSO—The minimum antibody profile signatures that capture the variation in viral rebound dynamics (DNA and days to rebound) was identified using the Least Absolute Shrinkage and Selection Operator (LASSO) method for feature reduction and regularization, which was implemented in a customized script in Matlab (Mathworks, Natick, Mass.). Ten-fold cross-validation was used to determine the optimal LASSO fit model with the lowest possible mean squared error (MSE) for prediction, and associated features were chosen as the minimum set of biomarkers. Principle component analysis (PCA) was used to assess the predictive ability of LASSO-selected biomarkers for classifying the participants with differential total HIV DNA changes and viral rebound.

### Example 1: Innate Effector Functions Induced by HIV gp120 Specific Antibodies

Since the half-life of IgG in peripheral blood in average is 3 weeks, we focused our analyses on PNB-induced effects at v9, rather than v6, in order to ensure that most possible IgG from before PNB treatment initiation to have vanished, decreasing the chance of baseline-IgG masking the effect of PNB on newly synthesized and potentially modified IgG. Besides the on-PNB treatment specific analyses, we also employed linear regression to calculate β-values, representing the slope of the regressed lines for each participant, to generate an assessment of changes in featured antibody profiles over the entire study period.

Of the different viral reservoir size measures, only changes in HIV DNA were consistently inversely associated with time to viral rebound, which is why we chose to relate our analyses to this (Data not shown).

During latency-reversal, potent enough to cause increases in plasma virus, HIV envelope proteins are likely transiently expressed on the surface of infected cells, rendering virus-expressing cells vulnerable to detection by antibodies that may direct a wide array of innate immune destructive activities. Among the potential antibody effector functions, we observed that antibody-dependent complement deposition (ADCD) as well as antibody-dependent cellular phagocytosis (ADCP) increased significantly following PNB treatment (FIG. 1B). Yet, the levels of ADCP, antibody-dependent cellular cytotoxicity (ADCC), and antibody-dependent Natural-Killer cell degranulation (CD107a) did not show any relation to the decline in the viral reservoir size, measured by HIV DNA (Data not shown).

In contrast, increases in antibody-dependent Neutrophil phagocytosis (ADNP) and ADCD were significantly correlated with decline in total HIV DNA levels (FIGS. 1C-1F), highlighting a unique relationship between specific antibody effector functions and reservoir control. Furthermore, classification of participants into “high” or “low” ADNPβ or ADCDβ also showed significant differences in HIV DNAβ, with significantly lower HIV DNAβ levels among the ADNPβ “high” population and a trend towards lower HIV DNAβ among the ADCDβ “high” group (FIGS. 1G and 1H), while this was not observed for any of the other effector functions (FIG. 1I). Notably, the ADNP levels, during PNB dosing, was also significantly correlated with longer time to viral rebound after treatment interruption (FIG. 1J). Together these data indicates that ADNP and ADCD, can be linked to changes in the HIV DNA reservoir and/or control of the replicating virus.

We next assessed whether the levels of HIV-specific antibodies accounted for the observed PNB-induced changes in reservoir influencing antibody-functions. While antibody levels remained unchanged over the entire study period (data not shown), the correlation analysis between effector functions and antibody titer revealed that while some effector functions indeed co-varied with antibody concentration, ADNP and ADCD functions were surprisingly independent of antibody concentration (FIG. 1K), suggesting that other modifications to antibodies, and likely in the antibody Fc-domain, must occur during PNB treatment resulting in alterations in ADNP and ADCD function.

### Example 2: Assessment of Fc-N-Linked Glycosylation of HIV-1 gp120 Specific IgG

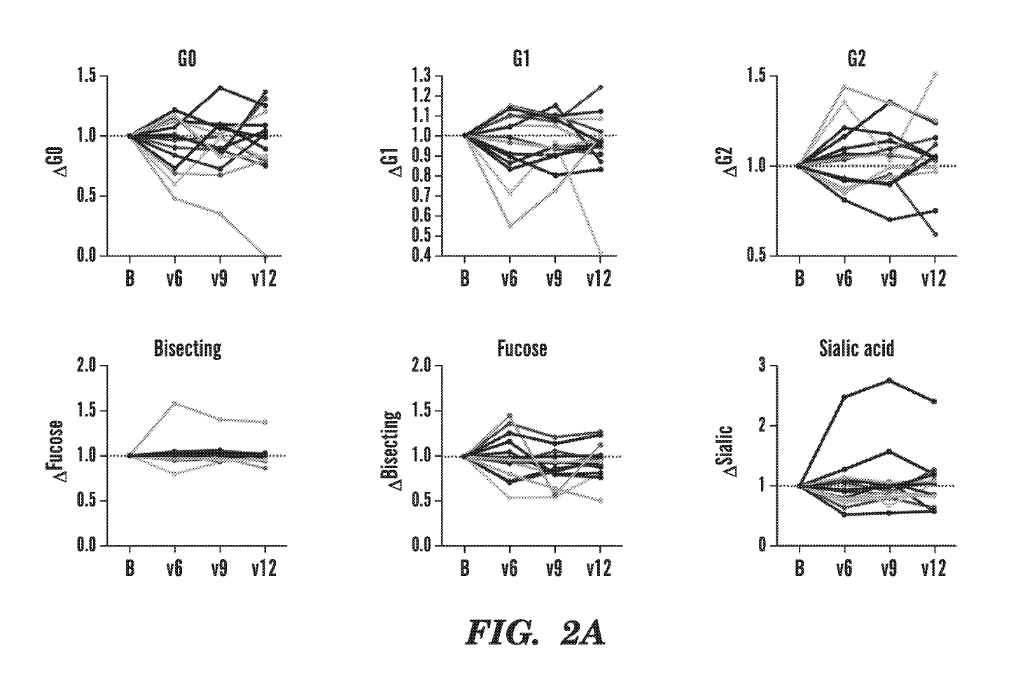

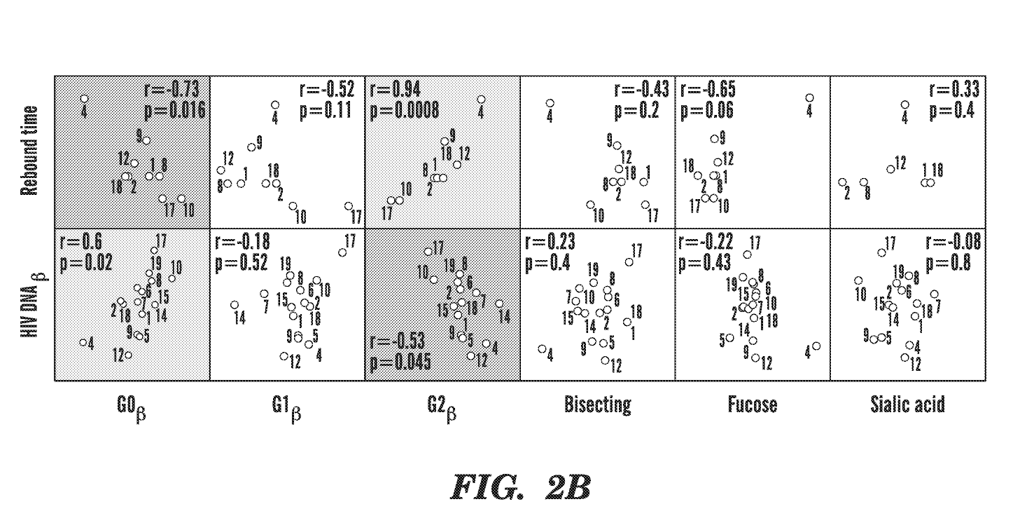

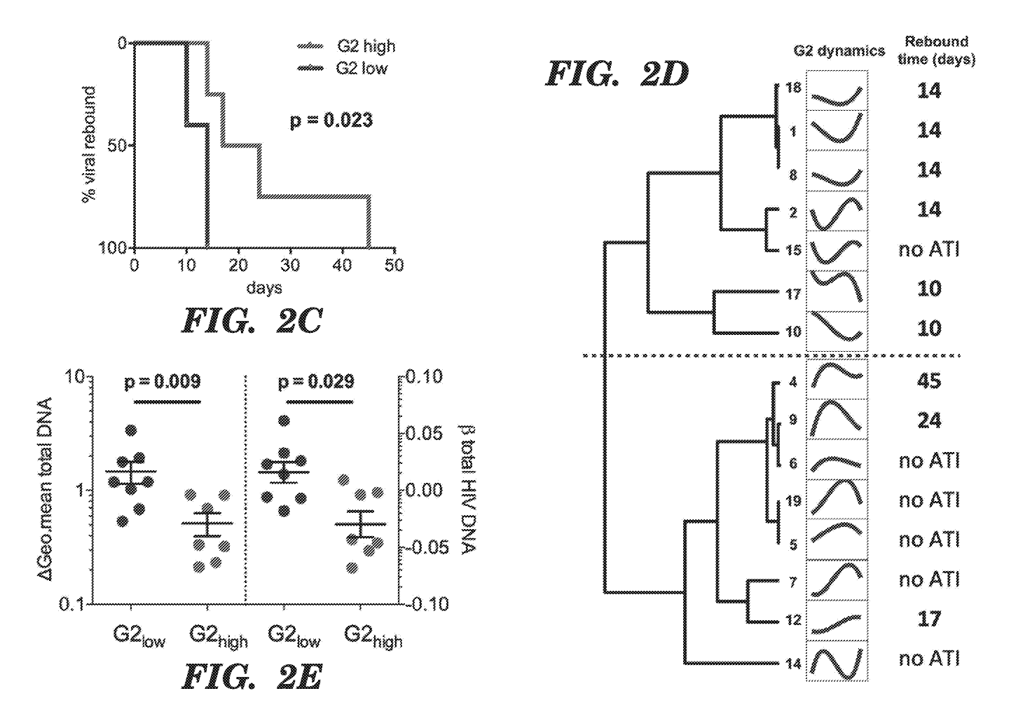

During an immune response, two modifications to the Fc-domain occur, aimed at tuning antibody functionality: 1) changes in antibody isotype/subclass levels and 2) changes in antibody n-glycosylation. As no differences were observed in IgG subclass distributions either over the study period (data not shown), we next investigated deeper into the biophysical structures and analyzed HIV gp120-specific IgG Fc-N-linked glycosylation by capillary electrophoresis. Surprisingly, while limited consistent changes in the glycan composition were observed among the donors after PNB treatment (FIG. 2A), changes in gp120-specific IgG-G2 glycoform (digalactosylated IgG) levels strongly correlated with time to viral rebound and inversely correlated with changes in total HIV DNA (FIG. 2B). In contrast, G0 glycoform levels (non-galactosylated IgG) showed the opposite significant patterns. Furthermore, the clustering of temporally dynamic changes in G2 glycoform levels was associated with viral rebound time, where the participants with initial G2 loss always exhibited the most rapid viral rebound time (FIG. 2C). Additionally, using the median G2β as a binary classifier, the group with the largest increase in G2 throughout the study also exhibited the longest time to viral rebound (FIG. 2D), as well as a significant decline in total HIV DNA over the study period, estimated by both change in geometric mean concentration from baseline (Δ) and HIV DNAβ (FIG. 2E).

### Example 3: Network Analysis Identifies Best Predictors

Because inter- and intra-individual dynamics of glycans and effector functions varied during treatment with PNB from donor-to-donor, all 198 features were integrated and analyzed to define the most predictive signatures of individual clinical endpoints including reservoir size and viral rebound kinetics. See Table 1.

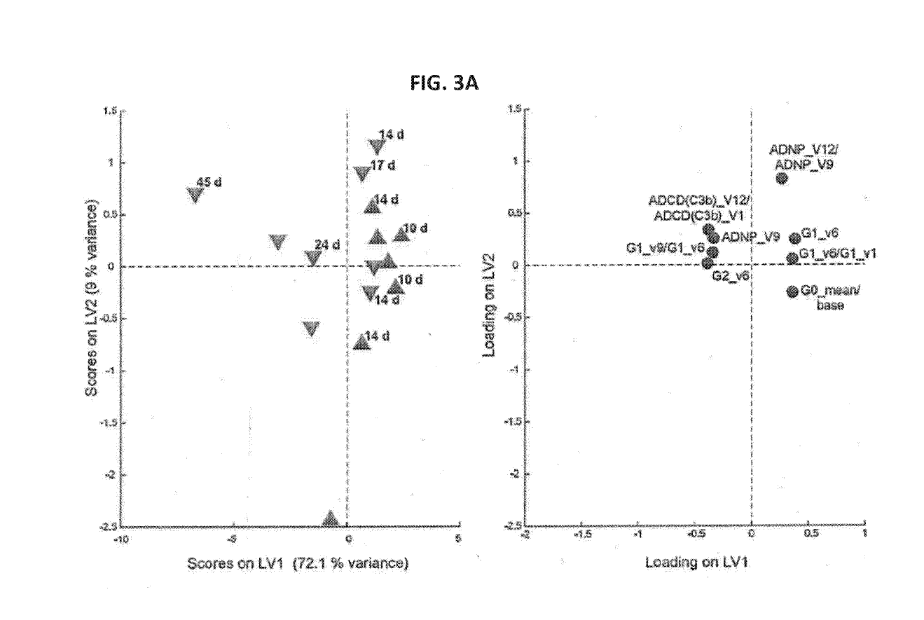

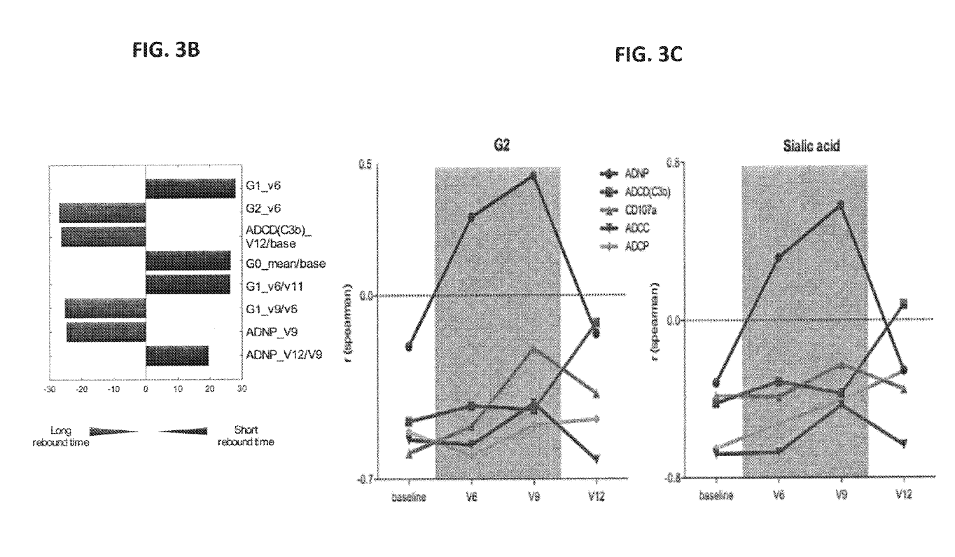

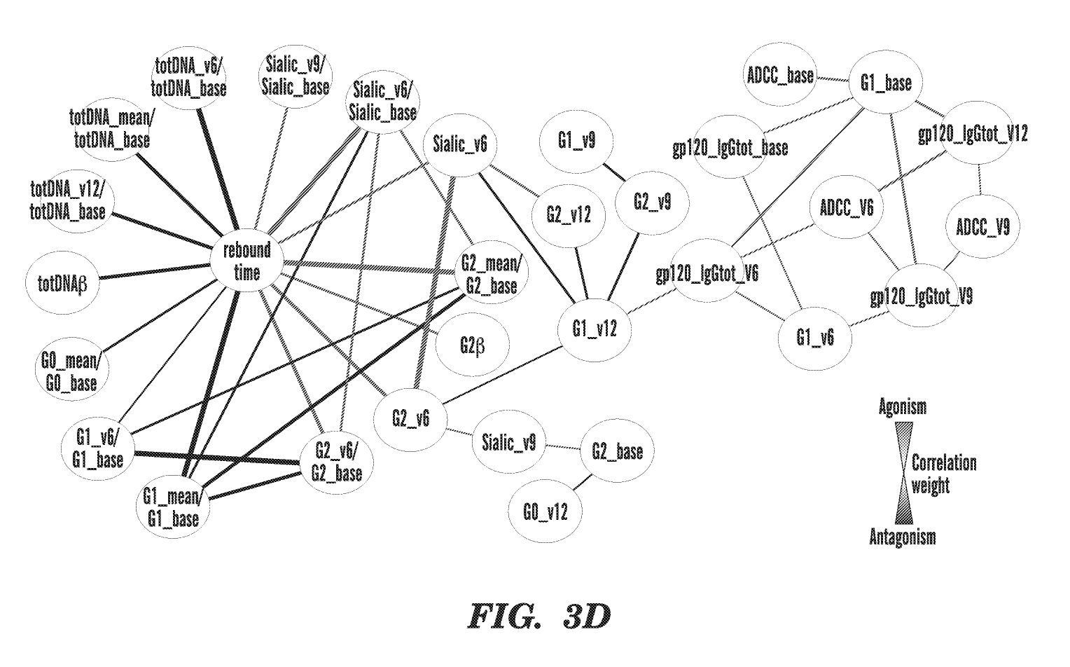

Yet, to identify the best predictors capturing the most differences of the outcomes within the cohort. LASSO (least absolute shrinkage and selection operator) (Tibshirani, 1997) was used to select the minimal set of the features from all the 198 features, that could separate the subjects based on viral rebound dynamics as well as HIV total DNA changes. Following feature reduction, complement binding (ADCD), neutrophil phagocytosis (ADNP) and IgG-G2 emerged again as the strongest predictors of DNA decline and slowest rebound times (FIGS. 3A and 3B).

As expected, viral rebound was inversely correlated with total HIV DNA, at the center of the correlation network, while agonized by G2 and sialic acid (FIG. 3D), further confirming the critical nature of these antibody signatures as biomarkers of PNB-driven reservoir control.

Furthermore, dynamic correlations between glycans and effector functions over time suggested a linkage between ADNP and the G2 and sialic acid glycoform levels, not observed for the other functional activities (FIG. 3C). ADNP, unlike all other effector functions, was the sole function that correlated positively with both G2 and sialic acid levels during PNB dosing. Since ADNP was found to be independent of antibody titer (FIGS. 1A-1K), this indicates that IgG-G2-glycoforms may partially drive ADNP function, which mechanistically could contribute to viral clearance upon latency reversal and decline in viral reservoir.

### Example 4: Glycan Substructures on HIV-1 gp120 Specific IgG is Associated with Both Function and Rebound Time

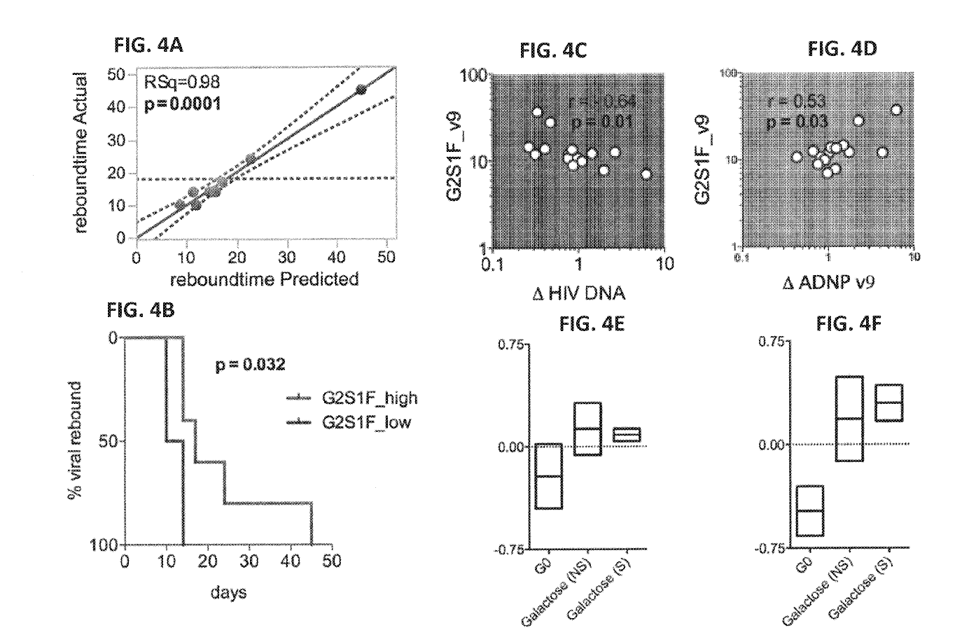

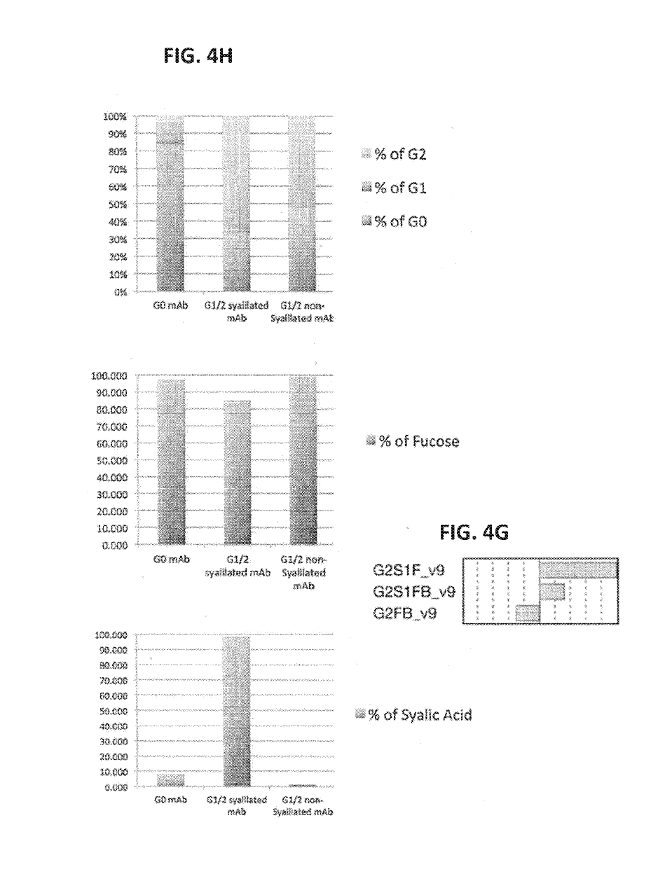

So far, our analyses were constricted to major groups of glycans. But considering that even minor differences in specific glycosylation profiles can have a huge impact on function (Peipp et al., 2008; Shields et al., 2002) we looked for linkages between glycosylation substructures and functional activities throughout the study. Multidimensional networks identified a linkage between some G2 substructures, ADNP and ADCC (data not shown). During PNB administration we identified three substructures, G2SF1, G2SF and G2S1FB, together predicting viral rebound time with a very high accuracy of 98%, using multiple linear regression. Of these three substructures G2S1F accounted for 92% of the variation (FIG. 4G). As in FIG. 2D, we classified G2S1F in high and low and observed almost identical patterns (FIG. 4B). With such a strong prediction, we now went back and analyzed the association of this particular substructure with the previous endpoints. In fact we found significant correlations with G2S1F during treatment with PNB and decline in HIV DNA, but also ADNP (FIGS. 4C and 4D), emphasizing that certain glycan in fact could be associated with both ADNP and decrease in the HIV reservoir.

To therefore determine whether G2 glycoforms contribute to ADNP activity, HIV-specific monoclonal antibodies were generated with distinct galactosylated-glycoforms (FIG. 4H). As predicted, the galactosylated variants of the HIV-specific antibody, PGT121, induced better neutrophil phagocytosis than the agalactosylated (G0) forms of the antibody (FIG. 4E). Since ADCD was also linked with decline in HIV DNA, fresh human complement was added to the ADNP assay in order to determine whether galactosylated glycoforms may harness both of the innate antiviral-processes to drive enhanced bioactivity. Interestingly, the G1/G2 antibodies drove more robust ADNP in the presence of complement (FIG. 4F), suggesting that galactosylated glycoforms may drive both enhanced neutrophil and complement activation that may synergistically contribute to reservoir control and clearance.

### Example 5: Predicting Viral Rebound in an Independent HIV-Cohort by Fc-Linked Glycan Structures

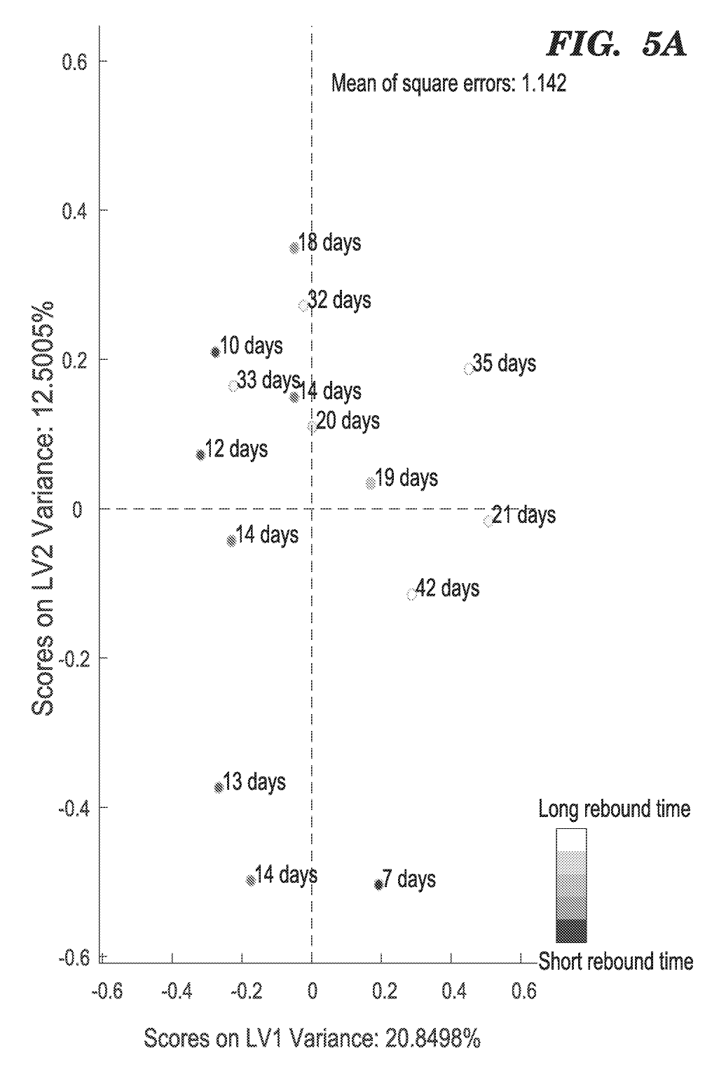

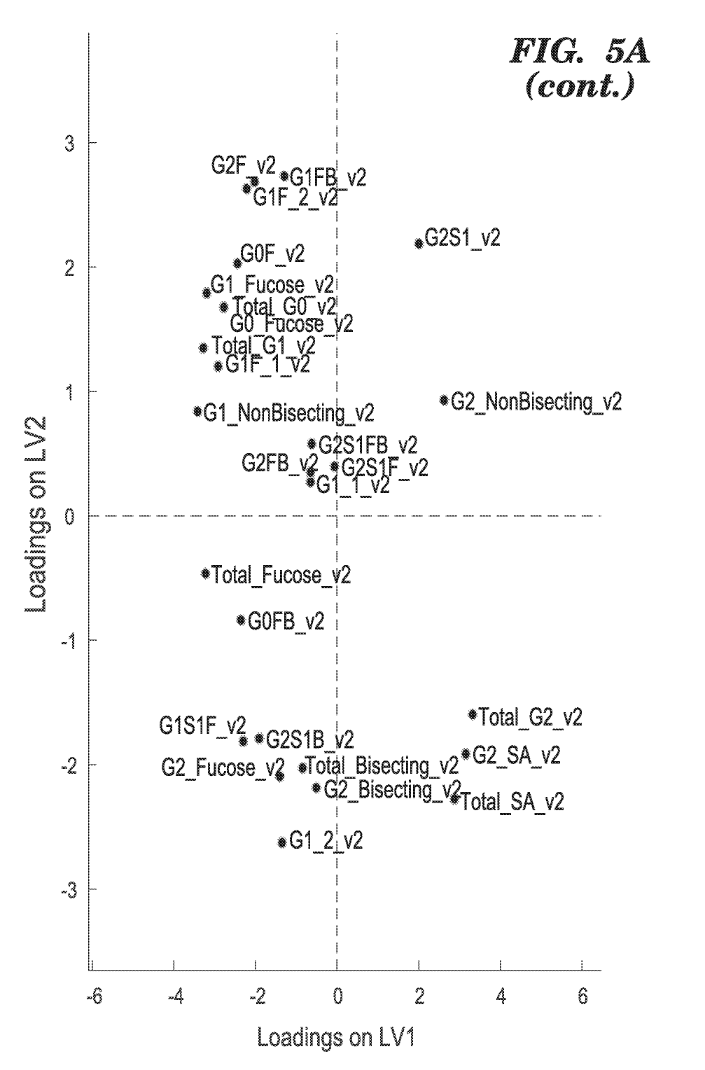

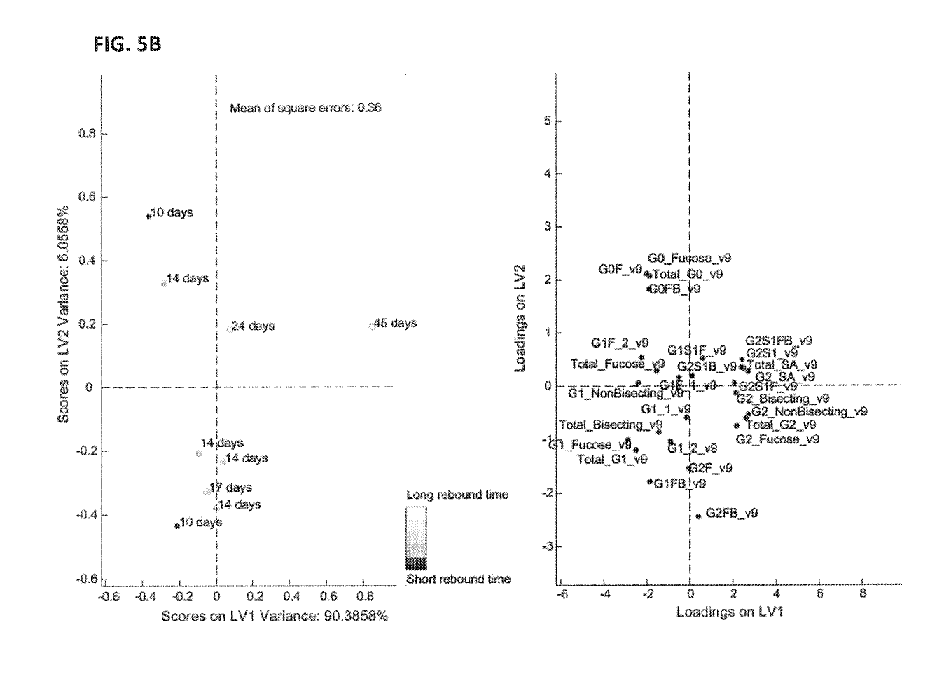

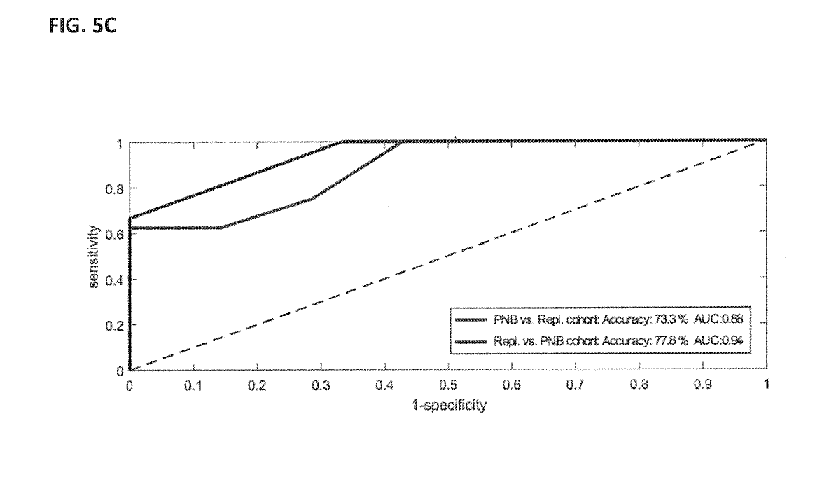

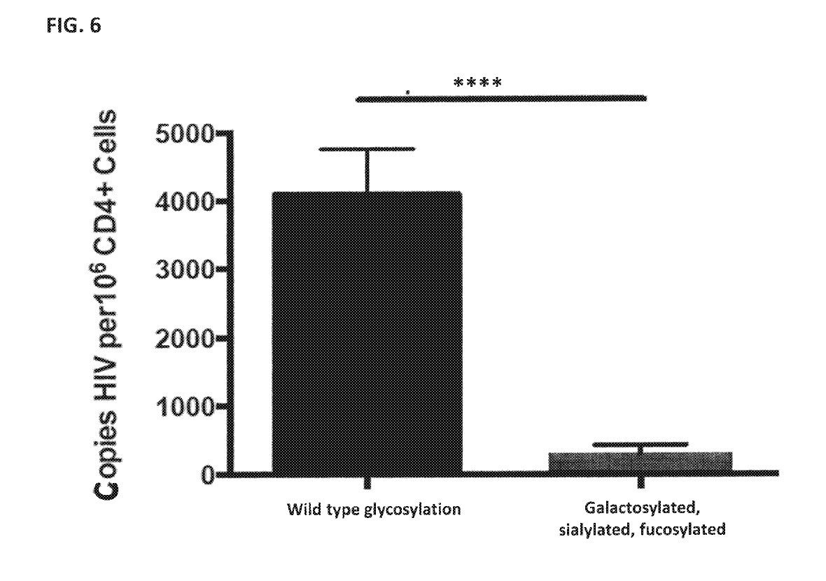

Observing these interesting patterns and associations, we next pursued determining whether antbody Fc-linked glycosylation patterns were simply a random association, despite of the strong linkages we discovered. Therefore, we investigated an independent HIV-1 cohort, receiving ART, but not exposed to latency disruption by an HDAC inhibitor (FIGS. 5A-5C).

We were indeed encouraged to discover, that certain glycan substructures again was associated with time to viral rebound in the replication cohort, once the patients were taken of ART (FIG. 5A). Then, applied to the PNB cohort, the same glycan structures were capable of explaining a remarkable 96% variance in rebound time (FIG. 5B). Asking how well these glycan structures in general were associated with rebound time for HIV infected, we consequently performed a cross-prediction analysis on the two independent cohorts. Intriguingly, the cross-prediction analysis found that the specific glycans in one HIV-cohort in fact could predict time to viral rebound in the other cohort, and vice versa, with as high as 75% accuracy (FIG. 5C). This point to the overall conclusion, that the Fc-inked glycans is very unlikely to be a random finding associated with viral rebound. Rather, it is more likely to be a naturally occurring phenomenon liked to viral control. Further, this effect seems to be enhanced when applying treatment with an HDAC inhibitor like PNB, although we cannot account for such a mechanism yet.

### REFERENCES

Ackerman, M. E. et al., (2016). Polyfunctional HIV-Specific Antibody Responses Are Associated with Spontaneous HIV Control. PLoS Pathog. 12, e1005315.
Archin, N. M. et al., (2012). Administration of vorinostat disrupts HIV-1 latency in patients on antiretroviral therapy. Nature 487, 482-485.
Chung, A. W. et al., (2014). Polyfunctional Fc-effector profiles mediated by IgG subclass selection distinguish RV144 and VAX003 vaccines. Sci. Transl. Med. 6, 228-238.
Chung, A. W. et al., (2015). Dissecting Polyclonal Vaccine-Induced Humoral Immunity against HIV Using Systems Serology. Cell 163, 988-998.
Connick, E. et al., (2007). CTL fail to accumulate at sites of HIV-1 replication in lymphoid tissue. J. Immunol. 178, 6975-6983.
Elliott, J. H. et al., (2014). Activation of HIV transcription with short-course vorinostat in HIV-infected patients on suppressive antiretroviral therapy. PLoS Pathog. 10, e1004473.
Finzi, D. et al., (1999). Latent infection of CD4+ T cells provides a mechanism for lifelong persistence of HIV-1, even in patients on effective combination therapy. Nat. Med. 5, 512-517.
Fuchs, S. P. et al., (2015). AAV-Delivered Antibody Mediates Significant Protective Effects against SIVmac239 Challenge in the Absence of Neutralizing Activity. PLoS Pathog. 11, e1005090.
Fukazawa, Y. et al., (2015). B cell follicle sanctuary permits persistent productive simian immunodeficiency virus infection in elite controllers. Nat. Med. 21, 132-139.
Gómez-Róman, V. R. et al., (2006). A simplified method for the rapid fluorometric assessment of antibody-dependent cell-mediated cytotoxicity. J. Immunol. Methods 308, 53-67.
Mahan, A. E. et al., (2015). A method for high-throughput, sensitive analysis of IgG Fc and Fab glycosylation by capillary electrophoresis. J. Immunol. Methods 417, 34-44.
Mahan, A. E. et al., (2016). Antigen-Specific Antibody Glycosylation Is Regulated via Vaccination. PLOS Pathog. 12, e1005456.
McAndrew, E. G. et al., (2011). Determining the phagocytic activity of clinical antibody samples. J. Vis. Exp. d, 1-5.
Olesen, R. et al., (2015). Innate Immune Activity Correlates with CD4 T Cell-Associated HIV-1 DNA Decline during Latency-Reversing Treatment with Panobinostat. J. Virol. 89, 10176-10189.
Peipp, M. et al., (2008). Antibody fucosylation differentially impacts cytotoxicity mediated by NK and PMN effector cells. Blood 112, 2390-2399.
Rasmussen, T. et al., (2014). Panobinostat, a histone deacetylase inhibitor, for latent-virus reactivation in HIV-infected patients on suppressive antiretroviral therapy: a phase 1/2, single group, clinical trial. Lancet HIV 1, e13-e21.
Shields, R. L. et al., (2002). Lack of Fucose on Human IgG1 N-Linked Oligosaccharide Improves Binding to Human Fcgamma Rill and Antibody-dependent Cellular Toxicity. J. Biol. Chem. 277, 26733-26740.
Sips, M. et al., (2016). Fc receptor-mediated phagocytosis in tissues as a potent mechanism for preventive and therapeutic HIV vaccine strategies. Mucosal Immunol.
Søgaard, O. S. et al., (2015). The Depsipeptide Romidepsin Reverses HIV-1 Latency In Vivo. PLoS Pathog. 11, e1005142.
Spivak, A. M. et al., (2014). A pilot study assessing the safety and latency-reversing activity of disulfiram in HIV-1-infected adults on antiretroviral therapy. Clin. Infect. Dis. 58, 883-890.
Tibshirani, R. (1997). The lasso method for variable selection in the Cox model. Stat. Med. 16, 385-395.
Zwirner, J. et al., (1989). Complement activation in human lymphoid germinal centres. Immunology 66, 270-277.

TABLE 1
Clustering of HIV gp120 specific antibody induced effector functions.
Left column shows distance between two effector function outputs.

| Distance | Leader | Joiner |
|---|---|---|
| 0.3391 | ADCC_v6 | ADCC_v9 |
| 0.4725 | MIP-1b_V12/MIP-1b_V1 | MIP-1b_β |
| 0.6584 | ADCP_v12/ADCP_v1 | ADCP_β |
| 0.7306 | ADCC_v1 | ADCC_v12 |
| 0.8770 | IFNy_V12/IFNy_V1 | IFN-y_β |
| 1.0578 | MIP-1b_V1 | IFNy_V1 |
| 1.1033 | ADCC_v1 | ADCC_v6 |
| 1.1084 | MIP-1b_V12 | 1FNy_V12 |
| 1.1152 | CD107_V12/CD107_V1 | CD107a_β |
| 1.3813 | ADCC_v12/ADCC_v1 | ADCC_β |
| 1.3932 | ADNP_V12 | ADNP_V12/ADNP_V9 |
| 1.5026 | MIP-1b_V6 | IFNy_V6 |
| 1.5078 | ADCD(C3b)_V6 | ADCD(C3b)_V9 |
| 1.5361 | CD107_V9/CD107_V6 | IFNy V9/IFNy_V6 |
| 1.5495 | ADCC_v12/ADCC_v9 | ADNP_V12/ADNP_V1 |
| 1.6772 | MIP-1b_V9/MIP-1b_V1 | MIP-1b_V12/MIP-1b_V1 |
| 1.7781 | ADCD(C3b)_V12/ADCD(C3b)_V1 | ADCD(C3b)_V12/ADCD(C3b)_V9 |
| 1.8024 | ADCP_v9/ADCP_v1 | ADCP_v9/ADCP_v6 |
| 1.8177 | ADCP_v9 | ADCP_v12 |
| 1.8837 | MIP-1b_V9 | IFNy_V9 |
| 1.8856 | CD107_V9/CD107_V1 | IFNy_V9/IFNy_V1 |
| 1.8868 | ADCD(C3b)_V12 | ADCD(C3b)_β |
| 1.9281 | CD107_V12/CD107_V9 | IFNy_V12/IFNy_V9 |
| 1.9591 | ADCP_v1 | ADCP_v6 |
| 1.9683 | MIP-1b_V6/MIP-1b_V1 | IFNy_V6/IFNy_V1 |
| 1.9934 | CD107_V12/CD107_V1 | MIP-1b_V9/MIP-1b_V1 |
| 2.0158 | MIP-1b_V9 | MIP-1b_V12 |
| 2.0458 | CD107_V6/CD107_V1 | MIP-1b_V6/MIP-1b_V1 |
| 2.0826 | CD107_V6 | CD107_V9 |
| 2.1355 | ADCD(C3b)_V1 | ADCD(C3b)_V6 |
| 2.2259 | ADCC_v9/ADCC_v1 | ADCC_v12/ADCC_v1 |
| 2.2620 | ADCD(C3b)_V6/ADCD(C3b)_V1 | ADCC_v9/ADCC_v6 |
| 2.2693 | ADNP_V6 | ADNP_V9 |
| 2.2763 | CD107_V1 | CD107_V12 |
| 2.3887 | ADCD(C3b)_V9/ADCD(C3b)_V1 | ADCD(C3b)_V9/ADCD(C3b)_V6 |
| 2.4995 | CD107_V12/CD107_V1 | IFNy_V12/IFNy_V1 |
| 2.5538 | MIP-1b_V6 | MIP- 1b_V9 |
| 2.5854 | ADCP_v6/ADCP_v1 | ADNP_β |
| 2.6228 | ADNP_V6/ADNP_V1 | ADNP_V9/ADNP_V1 |
| 2.6441 | CD107_V9/CD107_V6 | MIP-1b_V9/MIP-1b_V6 |
| 2.6929 | CD107_V12/CD107_V9 | MIP-1b_V12/MIP-1b_V9 |
| 2.7542 | ADCP_v1 | ADCP_v9 |
| 2.8391 | ADCP_v9/ADCP_v1 | ADCP_v12/ADCP_v1 |
| 2.8414 | ADCC_v12/ADCC_v9 | ADCP_v6/ADCP_v1 |
| 2.9566 | CD107_V1 | CD107_V6 |
| 3.0658 | ADCC_v6/ADCC_v1 | ADCC_v9/ADCC_vl |
| 3.1213 | CD107_V9/CD107_V6 | ADNP_V1 |
| 3.3655 | MIR-1b_V1 | MIP-1b_V6 |
| 3.3679 | CD107_V9/CD107_V 1 | CD107_V12/CD107_V1 |
| 3.8274 | ADCD(C3b)V12/ADCD(C3b)_V1 | ADNP_V6 |
| 3.9549 | CD107_V12/CD107_V9 | ADCP_v12/ADCP_v9 |
| 4.1092 | ADCD(C3b)_V6/ADCD(C3b)_V1 | CD107_V9/CD107_V6 |
| 4.2710 | ADCD(C3b)_V12 | ADCD(C3b)_V12/ADCD(C3b)_V1 |
| 4.3491 | ADCC_v1 | ADCP_v1 |
| 4.7149 | MIP-1b_V1 | ADNP_V12 |
| 4.7723 | ADCC_v12/ADCC_v9 | ADNP_V9/ADNP_V6 |
| 5.1259 | CD107_V1 | ADCC_v1 |
| 5.3852 | CD107_V6/CD107_V1 | CD107_V9/CD107_V1 |
| 5.5151 | CD107_V12/CD107_V9 | ADCP_v9/ADCP_v1 |
| 6.4041 | ADCC_v12/ADCC_v9 | ADNP_V6/ADNP_V1 |
| 6.9828 | ADCD(C3b)_V9/ADCD(C3b)_V1 | CD107_V12/CD107_V9 |
| 7.0753 | ADCD(C3b)_V6/ADCD(C3b)_V1 | ADCC_v6/ADCC_v1 |
| 7.3293 | ADCD(C3b)_V1 | CD107_V1 |
| 8.2186 | ADCD(C3b)_V12 | ADCD(C3b)_V9/ADCD(C3b)_V1 |
| 8.6019 | ADCD(C3b)_V6/ADCD(C3b)_V1 | MIP-1b_V1 |
| 9.2210 | CD107_V6/CD107_V1 | ADCC_v12/ADCC_v9 |
| 9.5937 | ADCD(C3b)_V12 | CD107_V6/CD107_V1 |
| 10.2765 | ADCD(C3b)_V12 | ADCD(C3b)_V6/ADCD(C3b)_V1 |
| 12.2109 | ADCD(C3b)_V1 | ADCD(C3b)_V12 |

2. Non-Limiting Examples of infections, diseases, or conditions for which synthesized engineered monoclonal antibodies can be generated for an dy-mediated immune response treatment, antibodies having the signature that correlated with positive outcome of the infection, disease, or condition.

| | Disease | Pathogen/target | Outcome |
|---|---|---|---|
| parasite | malaria | plasmodium falciparum or | days to parasite clearance |
| | | plasmodium vivax | |
| virus | merkell cell carcinoma | merkell virus | tumor remission/regression |
| | nasopharyngeal carcinoma | Ebstein Bar virus | remission/cure |
| | post-transplant lymphoproliferative | Ebstein Bar virus | control of virus |
| | disease (PTLD) | | |
| | burkitt's lymphoma | Ebstein Bar virus | remission/cure |
| | kaposi's sarcoma | HHV8 | remission/cure |
| | drug resistat CMV - CMV colitis, | cytomegalovirus | control of virus/viral eradication |
| | CMV hepatitis | | |
| | Genital/oral lesions | HSV1 and HSV2 | |
| | Hepatitits | hepattis B virus | cure/elimination of CCC-DNA |
| | cervical cancer | human papilloma virus | remission/tumor regression/cure |
| fungal | invasive fungal infection | aspegillus | eradciation/control |
| | invasive fungal infection | mucor | eradciation/control |
| bacteria | non-tubercuolosis mycobacterial | mycobacterium avium, | eradiation/control |
| | infections | mycabacterium intracellulare, | |
| | | mycobacteriu abscessus, | |
| | | mycobacterium kansasii, etc.. | |
| | extended spectrum beta-lactamase | gram-negative bacteria: e. coli, | eradiation/control |
| | producers | klbsiella, pseudomonas, | |
| | | neisseria gonorrhea | |
| autoimmune | Alzheimer's | tau or amyloid | regression/cure |
| | Multiple schlerosis | myelin | regression/cure |

What is claimed:

1. A method of screening for a humoral immunity signature for a clinical outcome of a disease or condition in a subject (a population), the method comprising:
a) providing at least one antibody sample obtained from the subject;
b) assaying a property or a characteristic of the at least one antibody sample in step (a);
c) determining a state of a clinical outcome in the subject at the point in time when the at least one antibody sample was collected in step (a);
d) correlating the property or characteristic of the at least one antibody sample obtained in step (b) with the state of the clinical outcome in the subject obtained in step (c); and
e) determining the presence of a positive or negative correlation which is indicative of a humoral immunity signature for the clinical outcome of the disease or condition.

2. (canceled)

3. The method of claim 1, further comprising using the signature to design a monoclonal therapeutic for use in the treatment of the disease or condition.

4. The method of claim 1, wherein the humoral immunity signature is a functional property, or a biophysical characteristic, or both a functional property and biophysical characteristic of an antibody in the subject.

5. The method of claim 1, wherein the state of clinical outcome of a disease or condition is selected from the group consisting of parasite load, pathogen load, virus load, bacteria load, fungal load, disease or condition symptom(s), tumor size, neuromuscular electrical conduction, cognitive performance, rebound viremia, size of viral reservoir, and length of protection (vaccine).

6. The method of claim 1, wherein the clinical outcome of a disease or condition is selected from the group consisting of faster rate parasite clearance, faster tumor remission, faster tumor regression, faster pathogen (e.g., parasites, virus, bacteria) clearance, faster elimination of clinical symptoms of the disease or condition, faster rate of pathogen eradication, faster rate of elimination of CCC-DNA, and slower rate of disease progression or symptoms.

7. The method of claim 1, wherein the subject has been treated for the disease or condition or is being treated for the disease or condition.

8. The method of claim 1, wherein the disease or condition is selected from the group consisting of malaria, merkell cell carcinoma, nasopharyngeal carcinoma, post-transplant lymphoproliferative disease (PTLD), burkitt's lymphoma, kaposi's sarcoma, drug resistant CMV-CMV colitis, CMV hepatitis, genital/oral lesions, hepatitis, cervical cancer, invasive fungal infection, non-tubercuolosis mycobacterial infection, extended spectrum beta-lactamase producer, Alzheimer's disease, multiple schlerosis, typhoid fever, and HIV infection.

9.-12. (canceled)

13. The method of claim 1, wherein the biophysical assays performed for the antibody samples comprises antibody isotyping subclass analysis, Fc-receptor binding assay, and glycosylation analysis of the Fc region of the antibody.

14. The method of claim 13, wherein the glycosylation analysis of the Fc region comprises analysis for galactosylation, sialation, bisecting GlcNAc-n-acetyleglucosamine, manosylation, n-acetylegalactosamine, glucosylation fucosylation, decreased bi-galactosylated (G2): decreased fucosylated G2 (G2F); decreased fucosylated G2 and bisected (G2FB); bisecting N-acetylglucosamine G0) G0B; fucosylated G0 with bisecting N-acetylglucosamine (G0FB); fucosylated G0 (G0F); and/or 1-galactose (G1).

15. The method of claim 1, wherein the functional property assay performed for the plurality of antibody samples is selected from the group consisting of Antibody dependent NK cell activation (ADNKA); Antibody-dependent cellular cytotoxicity (ADCC); Antibody-dependent cellular phagocytosis (ADCP); Antibody-dependent complement deposition (ADCD); Antibody-dependent neutrophil activation/phagocytosis (ADNP); Antibody dependent macrophage phagocytosis (ADMP); Antibody dependent dendritic cell (DC) phagocytosis (ADDCP); Antibody-dependent mucin binding (ADMB); Antibody-dependent eosinophil degranulation (ADED); and Antibody-dependent basophil degranulation (ADBD).

16. The method of claim 1, wherein the antibody is an IgG, IgA, or IgM antibody.

17. The method of claim 1, wherein the antibody is obtained from the plasma or serum of the subject.

18. The method of claim 1, wherein the property assay is performed with an antigen specific to the disease or condition.

19. The method of claim 1, wherein the antibody is antigen specific to the disease or condition.

20. (canceled)

21. A method of synthesizing engineered monoclonal antibodies for use with an antibody-mediated immune response to an infection, disease, or condition, the engineered monoclonal antibodies having a humoral immunity signature in the form of desired glycosylation in the Fc region of the antibodies, the signature correlates with a positive clinical outcome of infections, diseases, or condition, the method comprising:
a) providing an antibody sample obtained from the subject;
b) assaying a property or a characteristic of the antibody sample in step (a);
c) determining a state of a clinical outcome in the subject at the point in time when the antibody sample was collected in step (a);
d) correlating the property or characteristic of the antibody sample obtained in step (b) with the state of the clinical outcome in the subject obtained in step (c);
e) determining the presence of a positive or negative correlation which is indicative of a humoral immunity signature for the clinical outcome of the disease or condition; and
f) synthesizing engineered monoclonal antibodies having the identified signature.

22. A method of treating an infection, disease, or condition in a subject comprising administering an engineered monoclonal antibodies having a humoral immunity signature in the form of desired glycosylation in the Fc region of the antibodies, where the signature correlates with a positive clinical outcome of infections, diseases, or condition.

23. A method of predicting a HIV rebound viremia in a subject, the method comprising:
a. at a first time point, assaying a first blood sample obtained from the subject to determine a first level of the HIV glycoprotein 120-specific IgG-G2 glycoform antibody in the subject;
b. at a second time point, assaying a second blood sample obtained from the subject to determine a second level of the HIV glycoprotein 120-specific IgG-G2 glycoform antibody in the subject, wherein the second time point is after the first time point;
c. comparing the first level with the second level for indication of a downward trend or an upward trend of the antibody titer with the progression of time wherein a downward trend of antibody titer indicates the likelihood of a HIV rebound infection; and
d. administering to the subject an anti-retro viral agent when there is a downward trend of the antibody titer, wherein administration to the subject of the anti-retroviral agent treats the HIV infection.

24. The method of claim 23, wherein the subject has terminated an anti-retro viral therapy and there is undetectable HIV viral load serologically from the subject.

25. The method of claim 24, wherein the subject is treated with an agent to reactivate the latent HIV infection.

26. (canceled)

27. The method of claim 24, wherein the anti-retroviral agent is selected from the group consisting of a highly active anti-retro viral therapy (HAART), a nucleoside, a nucleoside reverse transcriptase inhibitor (NRTI), a non-nucleoside reverse transcriptase inhibitor (NNRTI), a nucleoside analog reverse transcriptase inhibitor (NARTI), a protease inhibitor, an integrase inhibitor, an entry inhibitor, a maturation inhibitor, and combinations thereof.

28. (canceled)
